(19) United States
(12) Patent Application Publication
(10) Pub. No.: US 2025/0144222 A1
(43) Pub. Date: May 8, 2025

(54) Bifunctional Compounds Targeting the Cation-Independent Mannose 6- Phosphate Receptor

(71) Applicant: NANOMEDSYN, Montpellier Cedex 5 (FR)

(72) Inventors: Marcel GARCIA, Prades le Lez (FR); Marie MAYNADIER, Saint Andre De Sangonis (FR); Ilaria BASILE, Saint Gely Du Fesc (FR); Khaled EL CHEIKH, Montpellier (FR); Morgane DAURAT, Montpellier (FR); Corentin GAUTHIER, Montpellier (FR)

(73) Assignee: NANOMEDSYN, Montpellier Cedex 5 (FR)

(21) Appl. No.: 18/838,161

(22) PCT Filed: Feb. 14, 2023
(86) PCT No.: PCT/FR2023/050196

§ 371 Date: Aug. 13, 2024

(30) Foreign Application Priority Data
Feb. 15, 2022 (FR) FR2201335

(51) Int. Cl. A61K 47/54; C07K 16/22; C07K 16/24; C07K 16/32

(52) U.S. Cl. CPC A61K 47/549; C07K 16/22; C07K 16/241; C07K 16/32

(57) ABSTRACT

New conjugates having a general formula (I). The conjugates include at least one mannose 6-phosphate analogue targeting the cation-independent mannose 6-phosphate receptor and an antibody or antibody fragment for binding a specific target antigen or target molecule. Also, a process for preparing the conjugates and their medical use, in therapeutic or diagnostic fields. The conjugates of formula (I) are particularly interesting for use as a medicine, particularly in a therapeutic treatment method in which a therapeutic antibody or a therapeutic antibody fragment is administered.

### FIELD OF THE INVENTION

The present invention falls within the field of therapeutic chemistry. It relates more particularly to bifunctional compounds comprising 1/at least one mannose 6-phosphate (M6P) analogue targeting the cation-independent mannose 6-phosphate receptor (CI-M6PR) and 2/an antibody or antibody fragment enabling the binding of a specific target antigen or target molecule. The invention also concerns the process for preparing said bifunctional compounds and their medical use, in therapeutic or diagnostic fields.

### BACKGROUND OF THE INVENTION

Most therapeutic molecules targeting disease-associated proteins neutralize the above mentioned target, thereby blocking the pathophysiological function of their target. However, therapeutic molecules often have little effect on the expression or degradation levels of the target. In addition, there are still many proteins that are not yet pharmacologically targeted, and whose deregulation and accumulation cause a pathological state.

In recent years, the targeted degradation of proteins involved in various diseases has emerged as a key strategy for achieving therapeutic efficacy. For the targeted degradation of proteins via the proteasome activation, degradation-inducing drugs are used, such as chimeras targeting proteolysis. However, the design of these chimeras is complex and cell permeability is limited. Moreover, as the ubiquitin-proteasome system is intracellular, the use of such chimeras cannot be applied to extracellular proteins.

A physiological pathway for protein degradation is represented by the endo-lysosomal pathway. Lysosomes are the main degradation compartments of eukaryotic cells. In addition to the proteasome, lysosomes can also degrade molecules other than proteins such as glycosaminoglycans, oligosaccharides and lipids. Molecules capable of directing pathological proteins to the lysosome for degradation have therefore been developed.

The cation-independent mannose 6-phosphate receptor (CI-M6PR), a 300 kDa transmembrane glycoprotein, represents the main transport and sorting pathway for the lysosomal enzymes carrying the mannose 6-phosphate (M6P) signal to lysosomes. The CI-M6PR also mediates endocytosis of M6P-containing extracellular ligands (Gary-Bobo et al., 2007). M6P-containing proteins, which differ from lysosomal enzymes and are internalized by means of the CI-M6PR-dependent transport, include granzyme B, a protease involved in cytotoxic T-cell-induced apoptosis, herpes simplex virus (HSV) and even leukemia inhibitory factor, a multifunctional protein that plays an important role in the formation of neurons, platelets and bone. The prerequisite for proteins to reach the lysosome is therefore the presence of M6P. However, M6P can be dephosphorylated by serum phosphatases, losing its ability to bind to CI-M6PR (El Cheikh K. et al., 2016). Some lysosomal enzymes have thus been modified to significantly increase the number of M6P residues, although these residues are still degraded by phosphatases (Zhu Y., et al., 2009).

M6P analogues have been synthesized in which the phosphate is replaced by other groups enabling recognition by CI-M6PR, without being degraded by phosphatases. M6P analogues refer to a compound which is slightly different from the natural M6P compound, but which has a high binding capacity to CI-M6PR. M6P analogues were proven to be effective in enhancing the therapeutic potential of a lysosomal enzyme produced in a baculovirus/insect cell expression system lacking the M6P signal (El Cheikh K. et al., 2016) and even of a lysosomal enzyme that already carries M6P (Basile I. et al., 2018).

The documents EP 2 448 600 B1 and EP 3 350 192 B1 describe M6P analogues, called AMFA (“Analogues of Mannose 6-phosphate Functionalized at the Anomeric position”), which present several advantages for the functionalization of glycoproteins, particularly for the functionalization of lysosomal enzymes. Indeed, the M6P analogues described in these documents are of small size, have low immunogenicity and can be easily grafted onto the lysosomal enzyme. These analogues can also be modulated in terms of both length and structure of the spacer arm, as well as in terms of the terminal reactive group of the spacer arm, depending on the desired type of linkage. In these documents, M6P analogues are more particularly used to internalize lysosomal enzymes into the lysosomes of the cell, so that said enzymes can play their physiological role.

PCT/US2019/067228 document described bifunctional molecules obtained by grafting a polypeptide onto an antibody, said polypeptide comprising a number of M6P analogues ranging from 20 to 90. The addition of this polypeptide to the antibody was proved to be effective in enhancing M6P receptor-mediated internalization of antigens into cultured cells. The fate of antibodies coupled to the polypeptide after cell entry is not analyzed.

PCT/IB2021/050922 document describes bifunctional compounds in which M6P analogues (M6 Pa) have been coupled to specific antagonist ligands for PCSK9 and FHR3 factors in order to decrease the extracellular concentrations of these factors. Cellular internalization of M6 Pa-antagonists complexed to targeted factors is followed by degradation in lysosomes. Coupling of M6 Pa analogues to antibodies is not considered in this document, and the fate of M6 Pa-antagonists after cellular entry is not analyzed.

The inventors have now developed new bifunctional compounds comprising at least one M6P analogue covalently linked to an antibody. These bifunctional compounds have the surprising and unexpected property of being able to be recycled to the outside of the cell after their initial cellular entry via the CI-M6PR pathway. This original and unexpected property makes it possible to envisage multiple reutilizations of the compound of the invention by cells, particularly to reduce/decrease the concentration of soluble or membrane extracellular antigens.

### SUMMARY OF THE INVENTION

According to a first aspect, the object of the present invention is a conjugate characterized in that it has the following general formula (I)

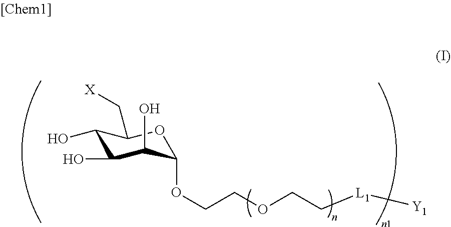

Wherein:
X is a phosphonate group —CH₂—P(O)(OZ)₂ or carboxylate group —CH₂—C(O)(OZ) where Z represents independently of each other H (hydrogen), Na (sodium), Li (lithium), K (potassium) or NH₄ (ammonium);
n is an integer ranging from 0 to 2;
L₁ is a radical selected from the group comprising *—O—N═**,

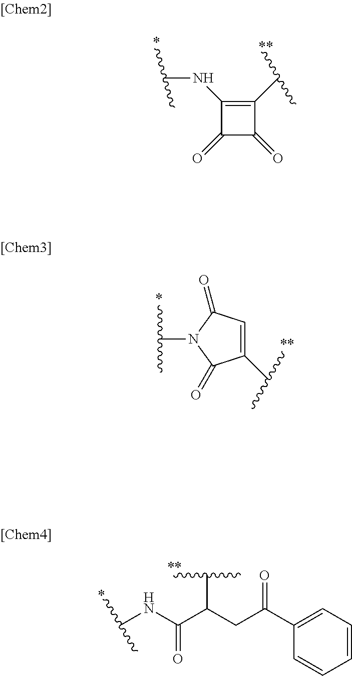 [SMILES: *Nc1c(*)c(=O)c1=O ; *C1=CC(=O)N(*)C1=O ; *NC(=O)C(*)CC(=O)c1ccccc1]

with * indicating the point of attachment of L₁ to the (O—CH₂—CH₂)ₙ group and ** indicating the point of attachment of L₁ to Y₁;
n₁ is an integer ranging from 1 to 20, preferably from 1 to 10,
Y₁ represents an antibody Y or an antibody fragment Y comprising at least one antigen-binding domain,
said Y₁ forming n₁ covalent bond(s) with L₁.

According to a second aspect, the object of the present invention is a process for preparing a conjugate of formula (I) as defined above.

According to a third aspect, the present invention relates to a conjugate of formula (I) for medical use, in therapeutic or diagnostic fields. It relates particularly to a conjugate of formula (I) for use in a method of therapeutic treatment which consists in administering a therapeutic antibody or a therapeutic antibody fragment.

### BRIEF DESCRIPTION OF THE DRAWINGS

Further features, details and advantages will become apparent from the detailed description below, and from an analysis of the appended drawings, in which:

FIG. 1 illustrates the extracellular recycling of conjugates of formula (I) of the invention. The M6P analogue is represented by the acronym AMFA. The conjugate of formula (I) can be called “AMFA-Antibody”. The antibody is represented by its Y shape, the AMFA by a small ball, and the antigen by a small cloud shape.

FIG. 2 illustrates the general synthesis scheme for conjugates of formula (I) in which n₁ is equal to 1, depending on the signification of the reactive chemical group L of the M6P analogue of general formula (II).

FIG. 2 point 1/illustrates the reaction between the oxy-amine function of the M6P analogue of formula (II) and the carbonyl function (—CHO) of antibody Y. Antibody Y is represented by Y₁′—CHO, with Y₁′ representing antibody Y without its functional group L′ equal to —CHO. Analogue (II) is designated AMFA1 when X is a phosphonate group and n is equal to 0 and AMFA2 when X is a carboxylate group and n is equal to 0.

FIG. 2 point 2/illustrates the reaction between the squarate function of the M6P analogue of formula (II) with the amine function (—NH₂) of the antibody Y. Antibody Y is represented by Y₁′—NH₂, Y₁′ representing the antibody Y without its functional group L′ equal to —NH₂. Analogues (II) are designated AMFA3 when X is a phosphonate group and n is equal to 0, and AMFA4 when X is a carboxylate group and n is equal to 0.

FIG. 2 point 3/illustrates the reaction between the maleimide function of the M6P analogue of formula (II) with the thiol function (—SH) of antibody Y. Antibody Y is represented by Y₁′—SH, Y₁′ representing antibody Y without its functional group L′ equal to —SH. The analogues (II) are designated AMFA5 when X is a phosphonate group and n is equal to 0 and AMFA6 when X is a carboxylate group and n is equal to 0.

FIG. 2 point 4/illustrates the reaction between the carbonylacrylic function of the M6P analogue of formula (II) and the thiol function of antibody Y. Analogues (II) are designated AMFA7 when X is a phosphonate group and n is equal to 0, and AMFA8 when X is a carboxylate group and n is equal to 0.

FIG. 3 illustrates the process for preparing intermediate 3: 2-bromoethyl 2,3,4-tri-O-trimethylsilyl-α-D-mannopyranoside, which will serve as a precursor for the synthesis of AMFAs of formula (II).

FIG. 4 illustrates the process for preparing the compound AMFA1 of general formula (II) from compound 3.

FIG. 5 illustrates the process for preparing the compound AMFA2 of general formula (II) from compound 3.

FIG. 6 illustrates the process for preparing compounds AMFA3 and AMFA5 of general formula (II) from compound 6.

FIG. 7 illustrates the process for preparing compounds AMFA4 and AMFA6 of general formula (II) from compound 1.

FIG. 8 illustrates the process for preparing compounds AMFA7 and AMFA8 of general formula (II) from compounds 16 and 21 respectively.

FIG. 9 shows the results obtained by SDS-PAGE electrophoresis and Western blots (WB) for mAb1, mAb2, mAb3 and mAb4 antibodies, alone or in combination with AMFA1 (mAb1-AMFA1, mAb2-AMFA1, mAb3-AMFA1 and mAb4-AMFA1). Antibodies before and after coupling with AMFA1 are detected either by protein staining with Coomassie Blue, or by recognition with anti-human IgG antibodies. The AMFA1s coupled to the antibodies are detected by polyclonal rabbit anti-AMFA1 serum (represented as “anti-AMFA” in the figure). Treatment of mAb1-AMFA1 with endonuclease H (PNGase) is also described, with detection by an anti-AMFA antibody.

FIG. 10 shows the affinity curves of mAb1 antibody or mAb1-AMFA1 conjugate (A), mAb2 antibody or mAb2-AMFA1 conjugate (B), mAb3 antibody or mAb3-AMFA1 conjugate (C), and mAb4 antibody or mAb4-AMFA1 conjugate (D), respectively, for their target molecules, namely human TNF-alpha (A and B), VEGF (C) and HER2 (D) antigens.

FIG. 11(A) shows data from flow cytometry analysis of the internalization of a non-specific control IgG antibody, mAb1 antibody and mAb1-AMFA1 conjugate together with a fluorescent anti-human IgG secondary antibody (Alexa647) in rhabdomyosarcoma cells (top graph), type-7 T-lymphocyte cells (middle graph) and breast cancer cells (bottom graph). [FIG. 11](B, C) show the entry of mAb1 antibody and mAb1-AMFA1 conjugate and mAb4 antibody and mAb4-AMFA1 conjugate previously coupled to Alexa647 into T lymphocytes (B) and SKOV3 cells (C), respectively.
** p<0.01 mAb1-AMFA1 versus mAb1 (Student's t-test) from 3 experiments.
*p<0.05 mAb4-AMFA1 versus mAb4 (Student's t-test) from 6 microscopic fields.

FIG. 12 shows cell fluorescence assay data from breast cancer cells (A) and rhabdomyosarcoma cells (B), treated with:
a non-specific control IgG antibody, the mAb1 antibody and the mAb1-AMFA1 conjugate with a fluorescent anti-human IgG secondary antibody (Alexa647) (breast cancer cells) (A),
mAb3 antibody and mAb3-AMFA1 conjugate with a fluorescent anti-human IgG secondary antibody (Alexa647) (rhabdomyosarcoma cells) (B).

This fluorescence assay enables to define the cellular internalization of the antibodies alone and of the conjugate of the invention.
** p<0.01 mAb1-AMFA1 versus mAb1 (Student's t-test).

FIG. 13(A) shows the internalization of Ab antibody or Ab-AMFA1 conjugate detected by confocal imaging in rhabdomyosarcoma cells (right) and breast cancer cells (left). Nuclei are labeled with Hoechst 33342 dye. Labeling of Ab or Ab-AMFA1 complexed to a fluorescent anti-human IgG secondary antibody (Alexa647) is indicated by arrows. The bottom 2 pictures concern the case where M6P excess was added to the culture medium to saturate the CI-M6PR binding sites of the cells. [FIG. 13](B, C) shows the internalization of the mAb1 antibody and the mAb1-AMFA1 conjugate in Jurkat T lymphocytes (B) and the reversion of this internalization by the addition of AMFA1 excess.
** p<0.01 mAb1-AMFA1 versus mAb1 (Student's t-test) n=6.

FIG. 14 shows data from the analysis of plasma samples from mice injected with mAb1 or mAb2 antibodies and the corresponding mAb1-AMFA1 or mAb2-AMFA1 conjugates. Antibody concentration in plasma is shown as a function of time after injection. The top graph (A) is for mice treated with mAb1 or mAb1-AMFA1 and the bottom graph (B) is for mice treated with mAb2 or mAb2-AMFA1.

FIG. 15 shows the detection by Western blots of mAb1 antibodies and mAb1-AMFA1 conjugates present in mouse plasma 21 days after intravenous (i.v.) injection of 4 mg/kg of mAb1 and mAb1-AMFA1. A control of injected mAb1-AMFA1 is added for comparison. Antibodies are detected by recognition with anti-human IgG antibodies, and the presence of AMFA coupled to antibodies is detected by anti-AMFA1 rabbit serum (represented as “anti-AMFA” in the figure).

FIG. 16(A) shows the cellular entry of human TNF-alpha into human T lymphocytes treated with 5 ng/mL human TNF-alpha and 20 ng/mL mAb1 or mAb1-AMFA1, for 1 h, 5 h and 17 h.
Cell lysates were analyzed by Western blot. Intracellular concentrations of human TNF-alpha were quantified as a function of time (hours) after treatment.
*p<0.01 mAb1-AMFA1+TNF-alpha versus mAb1+TNF-alpha (Student's t-test).

FIG. 16(B) shows the TNF-alpha concentrations measured in the culture media of the cells shown in (A) and corresponding to the different treatment times.

FIG. 17 shows the neutralization by mAb1 antibody or mAb1-AMFA1 conjugate of the anti-proliferative effect of human TNF-alpha on murine fibroblast-like cells treated with 5 ng/mL human TNF-alpha.
** p<0.01 mAb1-AMFA1 versus mAb1 (Student's t-test).

FIG. 18 shows the reversion of the mitogenic effect of VEGF in HUVEC cells treated with 50 ng/mL human VEGF and different concentrations of mAb3 or mAb3-AMFA1 for 72 h.

FIG. 19(A) shows the degradation of HER2 protein (human ERBB2) by protein immunocytochemistry (In Cell ELISA) in SK-BR-3 breast cancer cells treated with 10 or 100 nM mAb4 or mAb4-AMFA1 for 24 h. [FIG. 19](B) shows HER2 degradation in BT474 breast cancer cells treated with 5 nM mAb4 or mAb4-AMFA1 for 5 h.
*p<0.01 mAb4-AMFA1 versus mAb4 (Student's t-test) (n=2).

FIG. 20(A) shows the recycling capacity of mAb1 antibody and mAb1-AMFA1 conjugate in Jurkat T lymphocyte cells. Each antibody/conjugate is incubated for 5 h with the cells, then the culture medium is replaced by antibody-free medium. Cellular output of internalized antibody or conjugate is measured at 1 h and 5 h (n=2). [FIG. 20](B) shows the cellular exit of mAb1 antibody and mAb1-AMFA1 conjugate at 10 min and 30 min after 30 min of internalization in Jurkat T lymphocytes (n=4).
*p<0.05 mAb1-AMFA1 versus mAb1 (Student's t-test).

### DETAILED DESCRIPTION

### Conjugates of Formula (I)

As indicated, the object of the present invention is a conjugate of formula (I) as defined above. By “conjugate” in the sense of the invention is meant a compound comprising two parts linked together by a covalent bond. The first part of the conjugate represents at least one M6P analogue, while the second part of the conjugate represents an antibody or antibody fragment comprising at least one antigen-binding domain.

In the formula (I) defined above, the M6P analogue corresponds to the formula delimited by the large parenthesis. The integer n₁ indicates the number of M6P analogue(s) bound to the antibody or antibody fragment. Indeed, according to the invention, n₁ M6P analogue(s) can be covalently linked to an antibody or antibody fragment comprising at least one antigen-binding domain. These n₁ M6P analogues are linked to the antibody or antibody fragment via the L₁ radical.

As already indicated, an M6P analogue is a compound that differs from natural M6P but has a high binding capacity to CI-M6PR.

Throughout the application, M6P refers to mannose 6-phosphate and CI-M6PR to cation-independent mannose 6-phosphate receptor.

The conjugate of the invention is a bifunctional compound in the sense that it has a dual binding capacity, namely a capacity to bind to CI-M6PR (via the M6P analogue), and a capacity to bind to a specific target antigen or target molecule (via the antibody or antibody fragment).

The specific target antigen or target molecule refers to a membrane or extracellular molecule of therapeutic or diagnostic interest, in particular a molecule whose overexpression induces a pathological state or is associated with a pathological condition, or whose expression is involved in a pathological disorder.

In particular, the new bifunctional compounds of the invention make it possible to reduce/decrease the concentrations of extracellular or membrane target molecules in an individual, the said decrease being achieved by cellular endocytosis modulated by CI-M6PR, so that this reduction/decrease in concentrations is sufficient to no longer be in a pathological state.

As used herein, “extracellular or membrane target molecules” mean molecules of therapeutic or diagnostic interest, in particular molecules whose overexpression induces a pathological state or is associated with a pathological condition, or whose expression is involved in a pathological disorder.

In particular, the new bifunctional compounds of the invention make it possible to reduce/decrease the concentrations of a soluble extracellular antigen or of a membrane antigen in an individual, so that the individual no longer presents a pathological state.

In the general formula (I) of the conjugate of the invention, Y₁ has been defined as representing an antibody Y or an antibody fragment Y comprising at least one antigen-binding domain. In the following, for the sake of simplicity, antibody Y is understood to mean both the whole antibody Y and an antibody fragment Y comprising at least one antigen-binding domain, or an antibody-derived molecule comprising at least one of said antigen-binding domains.

Antibody Y refers to the free antibody, meaning that it does not form a bond with the M6P analogue.

Antibody Y₁ refers to antibody Y when it is covalently bound to the M6P analogue via the L₁ radical.

The free antibody Y can be represented as Y₁′-L′, with L′ representing a functional group or a reactive function carried by the antibody Y. In other words, Y₁′ represents the antibody Y without its functional group L′.

It is the functional group L′ of the antibody Y that will react with a functional group or reactive function carried by the M6P analogue to form a covalent bond between the antibody Y and the M6P analogue, thus obtaining the conjugate of formula (I) of the invention.

In the present application, the terms “functional group”, “reactive function” or “reactive chemical group” may be used interchangeably. Each of these terms designates a group carried by the antibody (or carried by the M6P analogue) capable of reacting with a group carried by the M6P analogue (or carried by the antibody).

The antibody is any type of antibody, of any isotype, from any species. It is a therapeutic or diagnostic antibody targeting a molecule of therapeutic or diagnostic interest, such as an anti-TNFα, anti-VEGF, anti-HER2 and anti-GFP antibody. The antibody is a heavy- and light-chain antibody, a single-chain antibody, a dimeric or multimeric, bispecific or multispecific antibody. The antibody is an immunoglobulin of the IgG type, in particular of the lgG1, lgG2, lgG3 or lgG4 subtype, of the IgE, IgD, IgA or IgM type.

“Antibody” includes monoclonal antibodies, humanized or fully human antibodies and chimeric antibodies from different species (human, mouse, rat, rabbit, goat, camelid, chicken, etc.), including human-mouse chimeric antibodies. Antibody fragments include Fab, Fab′, F(ab′)₂, Fv, scFv, Fabc or Fab fragments comprising a portion of the Fc region, and single-chain antibody fragments derived from camelid or shark immunoglobulins (V_{H}H single-domain antibodies (nanobodies) and V-NAR). Antibody-derived molecules include fusion proteins comprising a binding domain of an antibody to the target molecule, notably in the form of a Fe fusion protein; antibodies or fusion proteins having several binding domains corresponding to several target molecules, i.e. polyspecific antibodies or fusion proteins. The antibody can be further modified to confer a particular property in addition to its recognition of the target molecule, notably by conjugating the antibody to a therapeutically active molecule such as a cytotoxic molecule or a cell growth inhibitor, to a protease inhibitor, an element that increases the half-life of the antibody in the patient's blood, or an imaging molecule that enables the antibody and/or target molecule to be detected in the patient by the various techniques used in clinical practice (radiology, radiography, scintigraphy, MRI, endoscopy, scanner, etc.).

In the conjugate of formula (I) as defined above, Y₁ can more particularly be represented by the group L₂-Y′₁ in which Y′₁ represents the antibody Y linked to L₁ via the radical L₂ with L₂ chosen from the group comprising ═CH—, —NH— and —S—, said Y₁ can be represented by: ═CH—Y′₁, —NH—Y′₁ and —S—Y′₁.

More specifically, the L₂ radical is derived from the transformation of the functional group L′ carried by the antibody when the latter forms a covalent bond with the M6P analogue.

According to an advantageous embodiment of the invention, the conjugate of formula (I) is characterized in that n₁ is an integer equal to 1, and in that said conjugate is selected from the group comprising:

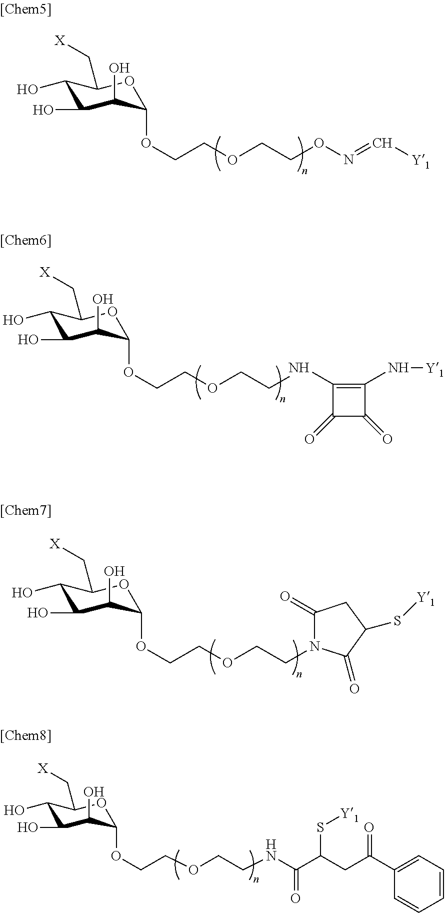

with X, n and Y′₁ as defined above.

According to yet another advantageous embodiment, the conjugate of the invention is characterized in that Y₁ represents a therapeutic or diagnostic antibody Y.

According to a particularly advantageous embodiment, the conjugate of the invention is more particularly characterized in that Y₁ represents an antibody Y selected from the group comprising monoclonal antibodies, chimeric antibodies, human or humanized antibodies, heavy- and light-chain antibodies, single-chain antibodies, bispecific or multispecific antibodies and nanobodies.

According to a further particularly advantageous embodiment, the conjugate of the invention is more particularly characterized in that Y₁ represents an antibody Y which is an immunoglobulin of the IgG type, in particular of the lgG1, lgG2, lgG3 or lgG4 subtype, or an immunoglobulin of the IgE, IgD, IgA or IgM type.

According to yet another embodiment of the invention, the conjugate of formula (I) as defined above can also be characterized in that it has an IC₅₀ affinity towards CI-M6PR ranging from 10⁻⁴ M to 10⁻⁹ M, and preferably ranging from 10⁻⁵ M to 10⁻⁸ M. This affinity can, for example, be measured by a competition method towards another ligand of CI-M6PR such as pentamannose 6-phosphate (according to Basile I. et al., 2018).

The conjugates of the invention are very interesting in that they enable cellular internalization of a target molecule, such as an antigen, via the endo-lysosomal pathway, this internalization being advantageously followed by extracellular release of the conjugates of the invention, thus enabling a new cycle of capture of the target molecule by the conjugate of the invention.

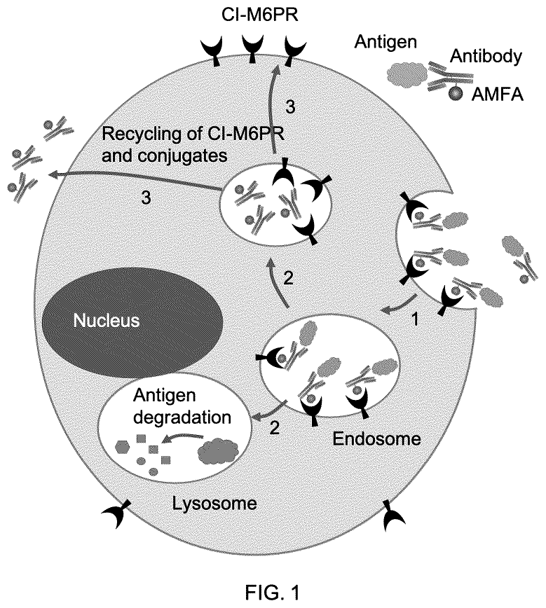

Extracellular recycling of the conjugates of the invention is illustrated in FIG. 1. The antigen-bound AMFA-antibody conjugate of the invention enters the cell endosome via CI-M6PR to deposit the antigen. The antigen is then directed to the lysosome, while the AMFA-antibody conjugate unexpectedly exits the cell. The conjugate thus released from the cell can be used to bind a new extracellular or membrane antigen.

This recycling capability was unexpected as previous examples in the literature using M6P analogues to internalize lysosomal enzymes (see for example EP 2 448 600 B1) indicated the presence of these bifunctional compounds (comprising M6P analogues linked to enzymes) in the endolysosomal pathway without extracellular recycling.

Similarly, in PCT/US2019/067228, bifunctional molecules associated with antigens are presented in lysosomes. This document provides no information on the possible extracellular recycling of bifunctional molecules. Thus, in all these studies, bifunctional compounds were described as being present and/or degraded in lysosomes along with their specific target. In the present invention, unexpectedly, with conjugates of formula (I), only the target molecule is degraded in lysosomes, while the conjugates are advantageously recycled in the extracellular space.

The conjugate of formula (I) is further characterized in that it has at least one of the following properties:
it is able to enter the endosome of the cell via its link with CI-M6PR to carry a soluble extracellular or membrane antigen;
it is able to leave the endosome of the cell after depositing the soluble extracellular or membrane antigen;
it enables the degradation in the lysosome of soluble or membrane extracellular antigens recognized by Y₁ after their cellular internalization;
it is recycled in the extracellular medium after its cellular internalization;

In a particularly advantageous embodiment, the conjugate of the invention exhibits each of the above-defined properties.

The originality of the conjugates of the invention lies in the fact that they do not destroy the natural ability of the antibody to leave the cell when it needs to. In fact, the coupling of M6P analogues with the antibody is carried out in such a way as not to create steric hindrance at the level of the antibody reactive functions, for example at the level of the part of the antibody interacting with specific antibody receptors, such as neonatal FcRn. This has the effect of not altering the extracellular recycling capabilities of the antibody when covalently linked to the M6P analogues described herein.

### Process for the Preparation of Conjugates of Formula (I)

According to another aspect, the object of the present invention is a process for preparing a conjugate of formula (I) as defined above, characterized in that:
an antibody Y or an antibody fragment Y comprising at least one antigen-binding domain with
n₁ compound(s) of general formula (II)

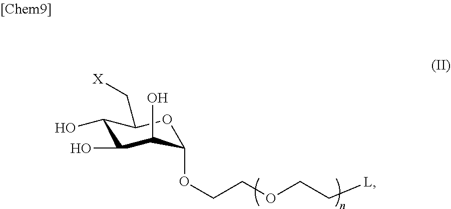

wherein X, n and n₁ are as defined above,
L is a reactive chemical group selected from the group comprising —O—NH₂,

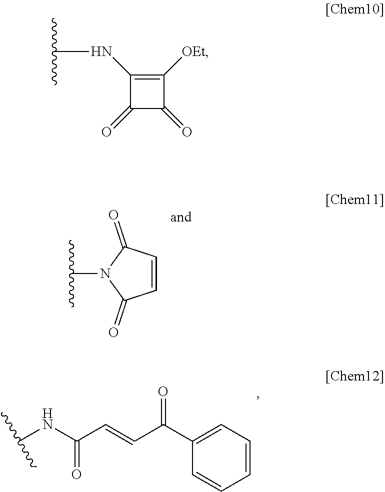 [SMILES: *Nc1c(OCC)c(=O)c1=O ; *N1C(=O)C=CC1=O ; *NC(=O)/C=C/C(=O)c1ccccc1]

said L group of compound (II) forming n₁ covalent bond(s) with n₁ functional group(s) carried by said antibody Y or antibody fragment Y.

The compound of formula (II) is an analogue of M6P. It is the reactive chemical group L carried by the M6P analogue of formula (II) which will react with the functional group L′ of the antibody Y to form a covalent bond between the antibody Y and the M6P analogue and thus obtain the conjugate of formula (I) of the invention. The L₂ radical is more particularly derived from the transformation of the functional group L′ carried by the antibody when the latter forms a covalent bond with the M6P analogue. Compounds of formula (II) are particularly interesting because of the simplicity of their spacer arm. As used herein “spacer arm” means the part located after the oxygen carried by the anomeric carbon of the cyclic structure of the M6P analogue and which comprises the reactive chemical group L, namely the part (CH₂)₂O—CH₂—CH₂)ₙ-L.

Indeed, the simplicity of the spacer arm structure contributes to the advantages of the conjugates of formula (I) of the invention. This simplicity of the spacer arm makes it easy to synthesize compounds of formula (II) and then to achieve a particularly interesting and effective coupling with an antibody. The synthesis of conjugates of formula (I) is thus efficient and easy to implement.

In the following, for the sake of simplicity, the L groups of the M6P analogue as defined above are referred to, in order of appearance, as “oxy-amine”, “squarate”, “maleimide” and “group comprising a carbonylacrylic function”, respectively.

Specific examples of M6P analogues of formula (II) are more particularly represented by the acronym “AMFA” in the present application.

According to an advantageous embodiment of the process of the invention, compound (II) is selected from the group comprising:

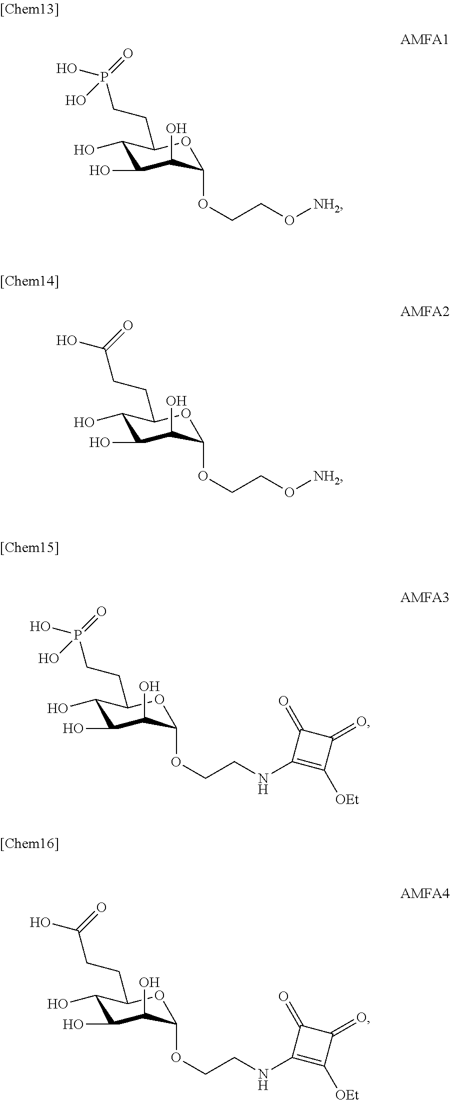

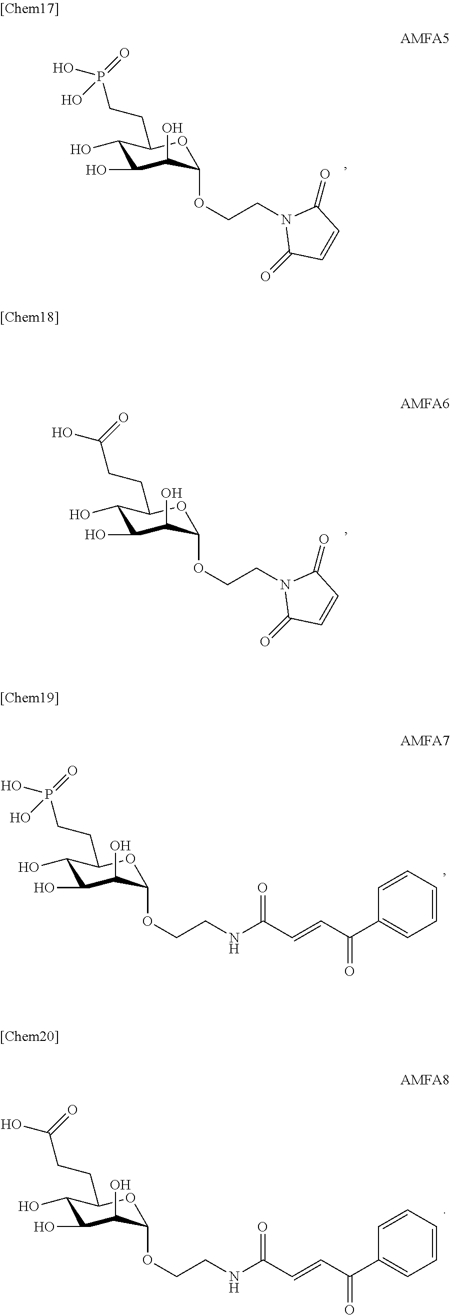

According to an advantageous embodiment of the process of the invention, the use of the above-described AMFA1 to AMFA8 enables a particularly interesting and effective coupling with an antibody as described above. Furthermore, as already indicated, the compounds of formula (II), and more particularly the AMFA1 to AMFA8 described above, are simple in terms of their chemical structure, notably due to the simplicity of their spacer arm. These compounds are easy to synthesize and then easy to couple with the antibody.

According to a particularly advantageous embodiment of the invention, the group L of the AMFA compound will bind to a carbonyl function previously generated on an oligosaccharide chain/part of the glycosidic part of the antibody. The aforementioned glycosidic part of the antibody is more particularly the one located at the Fc region of the antibody.

An example of a group L in the AMFA compound is the oxy-amine group.

According to another embodiment of the invention, the group L of the AMFA compound will bind to a suitable amino acid residue of the peptidic chain/part of the antibody.

Examples of suitable amino acid residues include an amine group from lysine or a thiol group from cysteine.

A squarate group, a maleimide group or a carbonylacrylic group are examples of groups L in the AMFA compound.

The terms “chain” or “part” used to refer to an oligosaccharidic chain or oligosaccharidic part of the antibody, or to the peptidic chain or peptidic part of the antibody, may be used interchangeably in the present application.

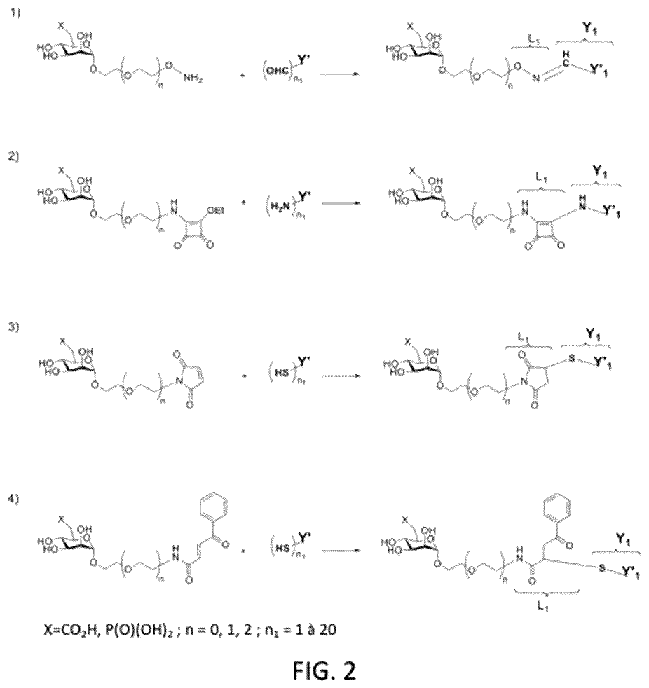

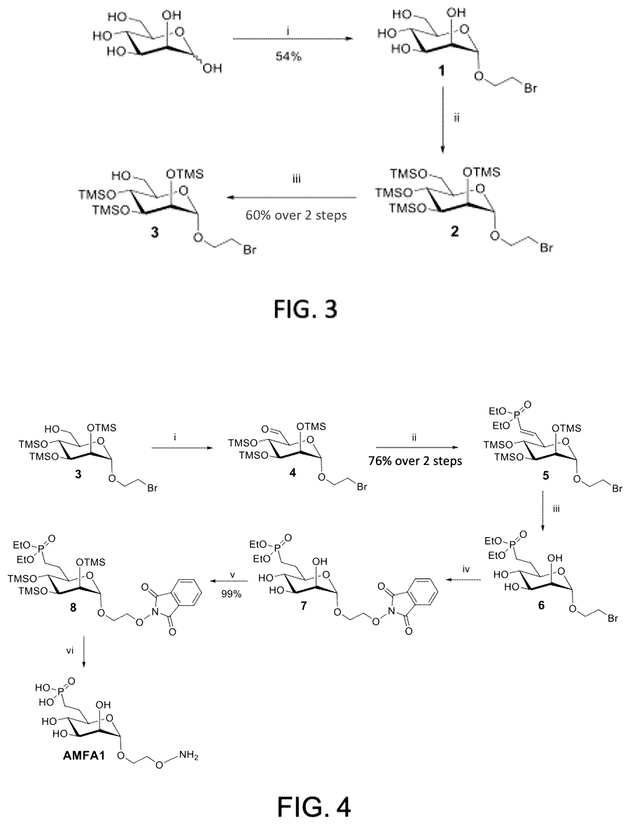

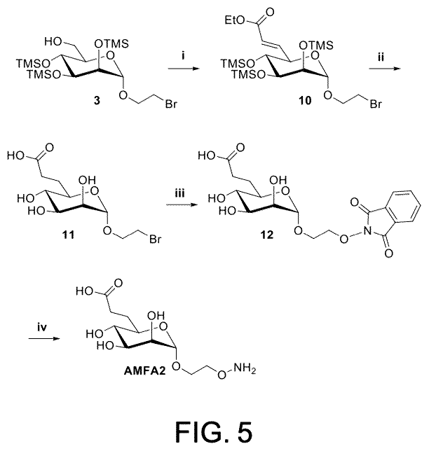

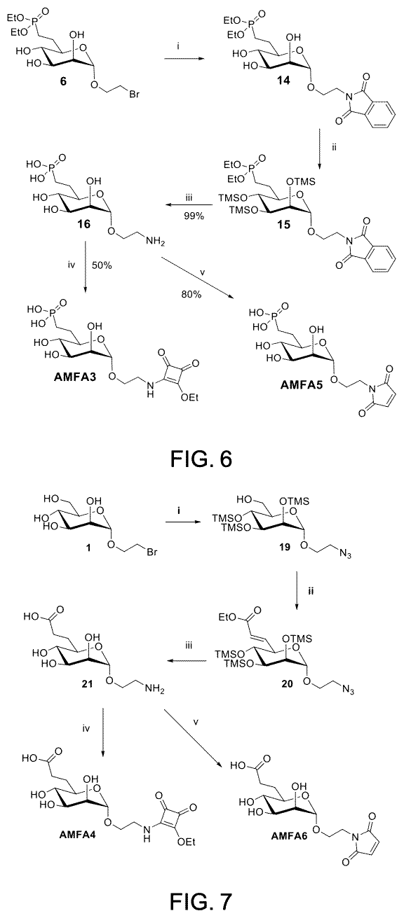

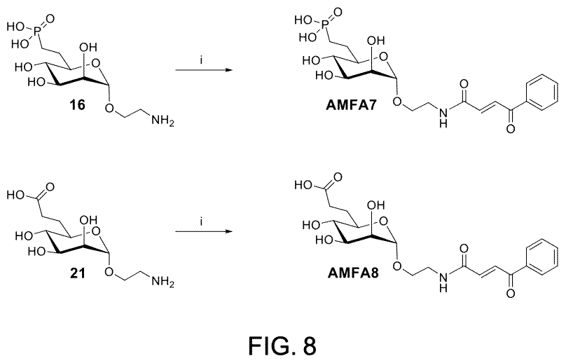

FIG. 2 illustrates the general synthesis scheme for the conjugates of the invention based on the respective definitions of the reactive chemical group L of the M6P analogue of general formula (II).

As shown in FIG. 2, point 1/, the carbonyl function (—CHO) of the antibody Y reacts with the oxy-amine function of the AMFA compound of formula (II). More specifically, this coupling mode takes place at the level of an oligosaccharidic chain of the glycosidic part located on the Fc part of the antibody.

This coupling mode is particularly advantageous in that it does not create any hindrance at the level of the other reactive functions of the antibody (such as, for example, the regions interacting with the receptors of the antibody), which advantageously enables the antibody to preserve its natural recycling properties to the outside of the cell and thus enables the conjugates of the invention to be recycled to the outside of the cell.

Regarding the coupling mode of the AMFA compound of formula (II) comprising the squarate group (see FIG. 2 point 2/), the latter intervenes at the level of a lysine residue of the peptide part of the antibody, namely more particularly at the level of the amine function.

Regarding the coupling of the AMFA compound of formula (II) with a maleimide group (see FIG. 2 point 3/), the latter occurs on a cysteine residue of the peptide part of the antibody, more specifically on the thiol function.

Regarding the coupling mode of the AMFA compound of formula (II) comprising a carbonylacrylic function (see FIG. 2 point 4/), this takes place at the level of a cysteine residue of the peptide part of the antibody, namely more particularly at the level of the thiol function.

These coupling modes are particularly advantageous because they create little or no hindrance at the level of the other reactive functions of the antibody (such as the antibody binding site for the FcRn receptors), which advantageously enables the antibody to preserve its natural properties and thus enables the conjugates of the invention to be recycled outside the cell.

### Use of Conjugates (I) in a Therapeutic Treatment Method or Diagnostic Method

The new bifunctional compounds can reduce/decrease the concentrations of soluble extracellular or membrane antigens in an individual, said reduction/diminishing being achieved by cellular endocytosis modulated by CI-M6PR. This property makes them particularly interesting for therapeutic use.

According to another aspect of the invention, a pharmaceutical composition is proposed, characterized in that it comprises:
a conjugate as defined above,
at least one pharmaceutically acceptable excipient.

The conjugate of the invention of formula (I) is present in a therapeutically effective amount in the composition of the invention.

By “therapeutically effective amount” we mean a dosage sufficient to produce a desired result, for example, an amount sufficient to obtain beneficial or desired therapeutic (including preventive) results, such as a reduction in the level of an element which extracellular or membrane expression or overexpression is responsible for a pathology or is involved in a pathological condition. An effective amount may require one or more administrations.

According to yet another aspect, the invention relates to a pharmaceutical composition comprising:
a therapeutically effective amount of the conjugate of formula (I),
at least one other therapeutically active agent,
optionally at least one pharmaceutically acceptable excipient.

The present invention also relates to a conjugate of formula (I) as defined above or a pharmaceutical composition as defined above, for medical use, of diagnostic or therapeutic type.

In this respect, the invention more particularly relates to a conjugate of formula (I) as defined above or a pharmaceutical composition as defined above, for use as a medicine.

A further object of the invention is a conjugate of formula (I) as defined above or a pharmaceutical composition as defined above, for use in a method of therapeutic treatment which comprises administering a therapeutic antibody or a therapeutic antibody fragment.

As already indicated, the ability to recycle extracellularly gives the conjugates of the invention particularly advantageous properties when used to treat pathologies in which the treatment consists of administering a therapeutic antibody or antibody fragment.

Indeed, administration of a conjugate of formula (I) enables a lower dose of therapeutic antibody to be used than if the therapeutic antibody was used alone (without being covalently linked to a M6P analogue). Indeed, when the therapeutic antibody is linked to a M6P analogue, its therapeutic efficacy is improved compared with the antibody used alone. For the same doses of antibody, the antibody linked to the M6P analogue is more effective than when used alone.

According to an advantageous embodiment of the invention, the use of a conjugate of formula (I) makes it possible to reduce the antibody doses usually used in a therapeutic treatment method involving the administration of antibodies, and thus to reduce the side effects associated with antibody treatment.

Examples of pathologies involving the administration of antibodies include those selected from the group comprising cancer, inflammatory diseases, autoimmune diseases, blood diseases, ophthalmological diseases, viral infections, allergic diseases and rare diseases.

Antibodies more particularly tested in the invention in coupling with AMFAs include anti-TNF-alpha, anti-VEGF and anti-HER2 antibodies. Anti-TNF-alpha antibodies are used to treat chronic inflammatory bowel diseases, Crohn's disease, ulcerative colitis and autoimmune diseases affecting the joints such as rheumatoid arthritis, axial spondyloarthritis/ankylosing spondylitis, psoriatic arthritis and psoriasis.

The anti-VEGF antibody is used clinically to treat neovascularization and tumor growth in many types of cancer, as well as ocular vascular disorders including neovascular age-related macular degeneration. As for the anti-HER2 antibody, it is notably used in the treatment of certain cancers, in particular breast cancers overexpressing the HER2 receptor.

A further object of the invention is a conjugate of formula (I) as defined above or a pharmaceutical composition as defined above, for use in a diagnostic method.

Thus, a conjugate of formula (I) covalently bound to an imaging molecule via the antibody Y₁ can be used to localize pathological lesions such as tumors or metastases, or other pathological areas to be treated. Examples of diagnostic methods include the diagnosis of diseases or conditions associated with increased CI-M6PR expression.

The conjugate of the invention or the pharmaceutical composition can be formulated in different pharmaceutical formulations (solid, semi-solid, liquid or gaseous form), such as tablets, capsules, powders, granules, ointments, solutions, injections, inhalations and aerosols. The pharmaceutical formulation may be in a liquid form, a lyophilized form or a liquid form reconstituted from a lyophilized form, where the lyophilized preparation must be reconstituted with a sterile solution prior to administration. Conjugates of the invention can be formulated into preparations for subcutaneous, intravenous or intravitreal injection by dissolving, suspending or emulsifying them in a solvent.

According to an advantageous embodiment, the invention relates to the conjugate of formula (I) or the pharmaceutical composition for use as defined above, characterized in that the conjugate or the pharmaceutical composition is in a form suitable for parenteral, intravenous or subcutaneous administration.

### EXAMPLES

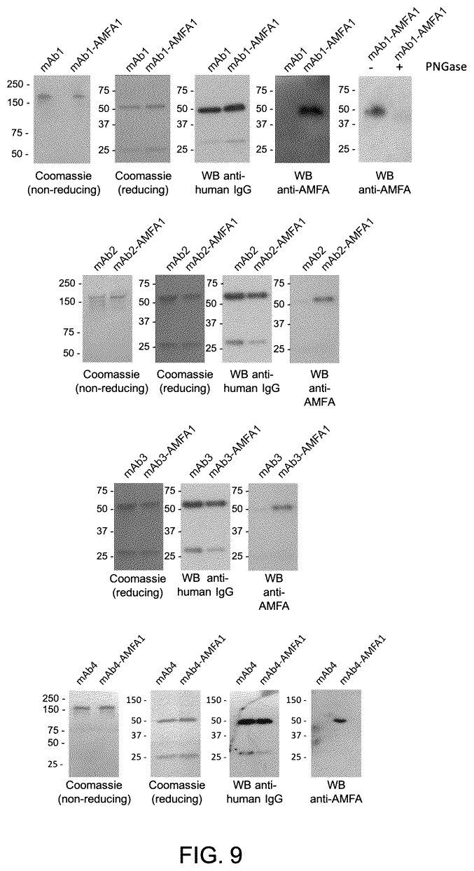

The synthesis of the conjugates (I) of the invention and the study of their biological effects are described in detail in the examples below, which refer to FIGS. 3 to 20. The M6P analogues of general formula (II) synthesized in the examples below are named AMFA1, AMFA2, AMFA3, AMFA4, AMFA5, AMFA6, AMFA7 and AMFA8 respectively.

Some of these AMFAs have been coupled with antibodies designated mAb1, mAb2, mAb3, mAb4 and Ab (see below).

### Example 1: Synthesis of AMFA1 to AMFA8 of General Formula (II)

### Preparation of Intermediate 3: 2-bromoethyl-2,3,4-tri-O-trimethylsilyl-α-D-mannopyranoside

The process for preparing intermediate 3 to be used as a precursor for AMFA synthesis is illustrated in FIG. 3, where the conditions and reagents for steps (i) to (iii) are as follows:
(i) 2-bromoethanol, triethylamine, CH₂Cl₂, 3.5 h, 80° C.; (ii) TMSCl, Et3N, CH₂Cl₂, 20 h, RT; (iii) K₂CO₃, MeOH, 15 min, 0° C.

The starting compound was D-mannose onto which 2-bromoethanol was introduced at the anomeric position using Amberlite IR120 resin previously reactivated to form compound 1. Compound 1 was persilylated with trimethylsilyl chloride in the presence of triethylamine to form intermediate 2. Position 6 of intermediate 2 was selectively deprotected by potassium carbonate in catalytic amounts in methanol to lead to compound 3 in 60% yield over two steps (see FIG. 3).

### Preparation of Compound AMFA1

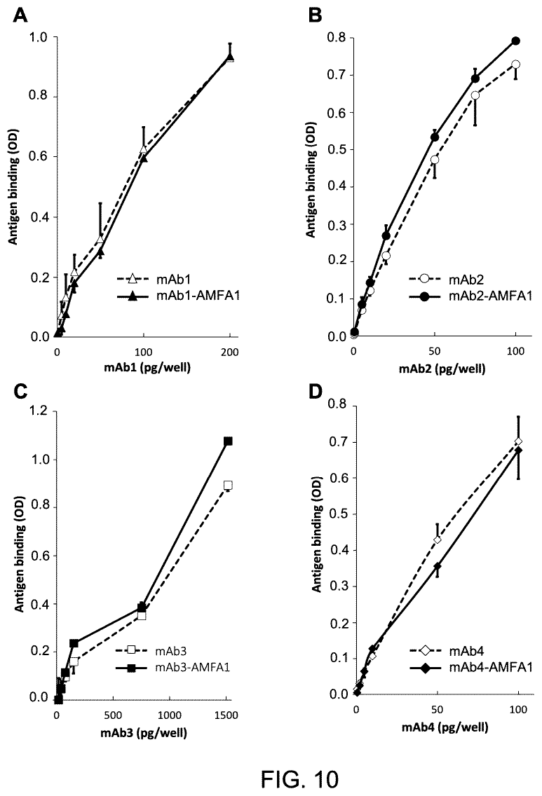

The process for preparing the compound AMFA1 from compound 3 is illustrated in FIG. 4, where the conditions and reagents for steps (i) to (vi) are as follows: (i) Dess-Martin periodinane 0.3 M, CH₂Cl₂, RT, 1 h; (ii) tetraethyl methylenediphosphonate, NaH, THF, 1 h, RT; (iii) Pd/C, Et₃SiH, MeOH, 2 h, RT. (iv) N-hydroxyphthalimide, NaH, DMF, 22 h, 60° C.; (v) TMSCl, Et₃N, CH₂Cl₂, 15.5 h, RT; (vi)-a) TMSCl, NaI, CH₃CN, 50 min, 35° C.; (vi)-b) N₂H₄·H₂O, MeOH, 15.5 h, RT.

Alcohol 3, obtained as described above, is oxidized to aldehyde using Dess-Martin's periodinane to form compound 4. This key intermediate provides access to the various AMFAs of formula (II).

To synthesize compound AMFA1, aldehyde 4 is reacted with tetraethyl methylenediphosphonate, previously deprotonated with sodium hydride, to form compound 5 in 76% yield over two steps. In a catalytic hydrogenation step, in the presence of palladium on charcoal and for which hydrogen is generated in situ by progressive addition of triethylsilane, the double bond is reduced and the trimethylsilyl groups are hydrolyzed. Compound 6 is thus obtained in quantitative yield without purification. The bromine atom is then substituted with N-hydroxyphthalimide to form intermediate 7 in 61% yield. The alcohol functions are then protected with trimethylsilyl chloride to give compound 8 in quantitative yield. The resulting unpurified intermediate 8 is then reacted with trimethylsilyl chloride and sodium iodide to form the bis(trimethylsilylated)phosphonate by a Rabinowitz-type reaction. This intermediate is then converted to phosphonic acid by the action of hydrazine monohydrate in methanol, which also displaces the trimethylsilyl groups present on the secondary alcohols of the sugar and reacts with the phthalimide to unmask the amine function. In a single step, we thus obtain the compound AMFA1 deprotected on the phosphonate part, on the sugar alcohols and bearing the aminooxy function at the anomeric position with a yield of 85% over two steps (FIG. 4).

### Preparation of Compound AMFA2

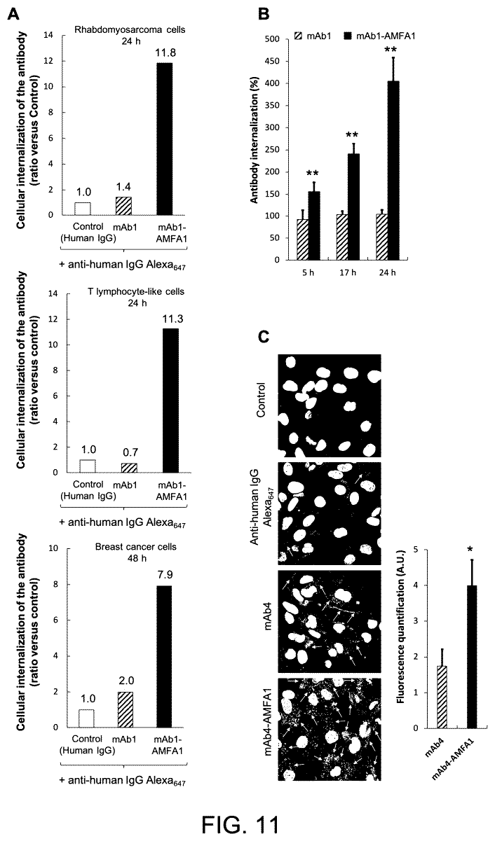

The process for preparing the compound AMFA2 from compound 3 is illustrated in FIG. 5, where the conditions and reagents for steps (i) to (iv) are as follows: (i)-a) Dess-Martin periodinane 0.3 M, CH₂Cl₂, RT, 1 h; (i)-b) Triethyl phosphonoacetate, NaH, THF, 20 min, RT, 73% (2 steps); (ii)-a) Pd/C, Et₃SiH, MeOH, 2 h, RT; (ii)-b) NaOH 1 N, THF/H₂O, 2 h, RT; (iii) N-hydroxyphthalimide, NaH, DMF, 20 h, 60° C.; (iv) N₂H₄·H₂O, MeOH, 15.5 h, RT.

The AMFA2 compound is synthesized following the synthetic routes described in FIG. 5. One of the main steps in the synthesis of this saccharide ligand was the introduction of the carboxylate function on the side chain of the mannose fragments. As in previous syntheses, this functionalization is achieved by a Dess-Martin oxidation of intermediate 3 in position 6 followed by a Horner-Wadsworth-Emmons reaction using triethyl phosphonacetate anion. In this way, the unsaturated carboxylate 10 is obtained. After reduction of the double bond and hydrolysis of trimethylsilyl protecting groups on the hydroxyl functions, the function carboxylate was saponified to give intermediate 11. The bromine was then substituted by hydroxyphthalimide to give intermediate 12. Demasking of the amine function by hydrazinolysis leads to the fully deprotected compound AMFA2 (FIG. 5).

### Preparation of Compounds AMFA3 and AMFA5

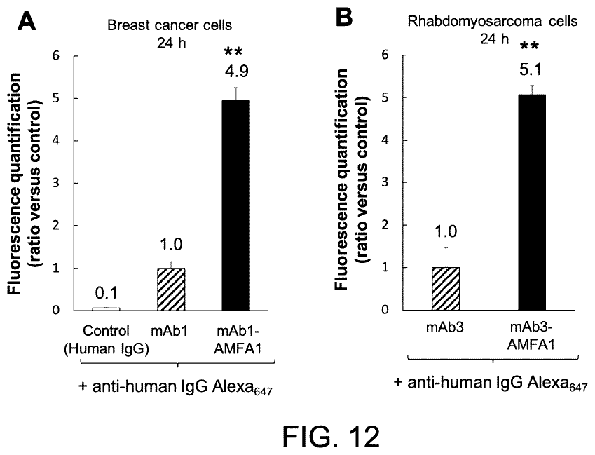

The process for preparing compounds AMFA3 and AMFA5 from compound 6 is illustrated in FIG. 6, where the conditions and reagents for steps (i) to (v) are as follows: (i) Dess-Martin periodinane 0.3 M, CH₂Cl₂, RT, 1 h; (ii) tetraethyl methylenediphosphonate, NaH, THF, 1 h, RT; (iii) Pd/C, Et₃SiH, MeOH, 2 h, RT. (iv) potassium phthalimide, DMF, 22 h, 60° C.; (v) TMSCl, Et₃N, CH₂Cl₂, 15.5 h, RT; (vi)-a) TMSCl, NaI, CH₃CN, 50 min, 35° C.; (vi)-b) N₂H₄·H₂O, MeOH, 15.5 h, RT; (vii) Diethyl squarate, Et₃N, EtOH/H₂O, 30 min; (viii) N-(methoxycarbonyl)maleimide, NaHCO₃ saturated solution, 30 min, 0° C.

To synthesize compounds AMFA3 and AMFA5, the bromine atom of compound 6 is substituted with potassium phthalimide to form intermediate 14 in 74% yield. The alcohol functions are then protected with trimethylsilyl chloride to give compound 15 in quantitative yield. Intermediate 15 thus obtained, without purification, is then reacted with trimethylsilyl chloride and sodium iodide to form the bis(trimethylsilylated)phosphonate by a Rabinowitz-type reaction. This intermediate is then converted to phosphonic acid by the action of hydrazine monohydrate in methanol, which also displaces the trimethylsilyl groups present on the secondary alcohols of the sugar and reacts with the phthalimide to unmask the amine function. In a single step, compound 16 deprotected on the phosphonate moiety, on the alcohols of both sugars and bearing the amine function in the anomeric position is obtained in a yield of 65% over two steps. Compound 16 was then reacted with either diethyl squarate to form the product AMFA3 in 50% yield, or with N-(methoxycarbonyl)maleimide to form the compound AMFA5 in 80% yield (FIG. 6).

### Preparation of Compounds AMFA4 and AMFA6

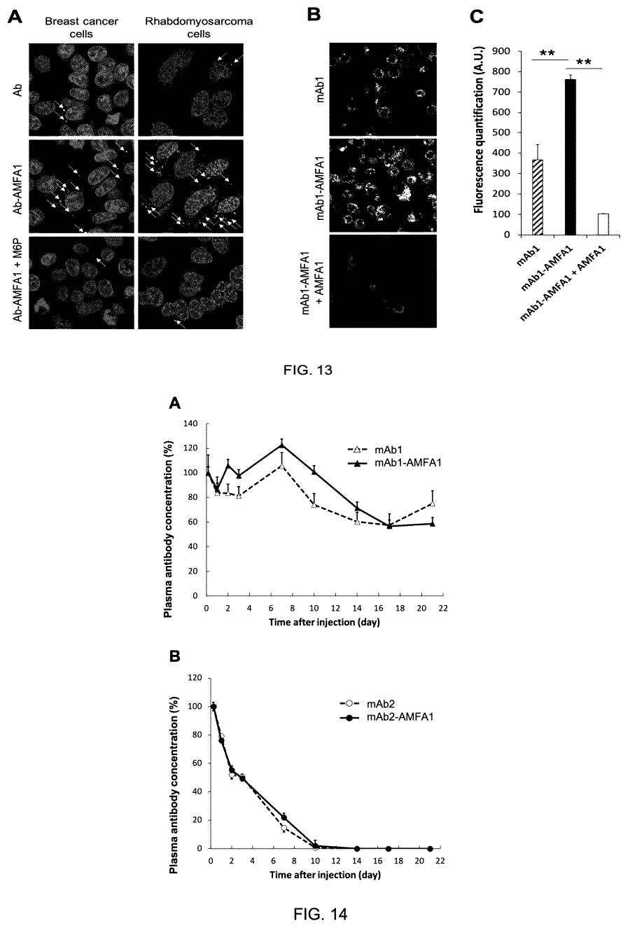

The process for the preparation of compounds AMFA4 and AMFA6 from compound 1 is illustrated in FIG. 7, where the conditions and reagents for steps (i) to (v) are as follows: (i) NaN₃, DMF, 20 h, RT; TMSCl, Et₃N, CH₂Cl₂, 20 h, TA; K₂CO₃ cat, MeOH, 50 min, 0° C., 38% (3 steps); (ii)-a) Dess-Martin's periodinane, CH₂Cl₂, 3 h, RT, (ii)-b) Triethylphosphonoacetate, NaH, THF, 2 h, RT, 73% (2 steps); (iii)-a) HCl 0.5 N, THF, 30 min, RT, (iii)-b) NaOH 0.1 N, 30 min, RT, 99%, (iii)-c) H₂, Pd/C, EtOH/H₂O, 1.5 h, RT, 99%; (iv) diethyl squarate, EtOH/H₂O, 40 min, RT, 34%; (v) N-(methoxycarbonyl)maleimide, NaHCO₃ saturated solution, 30 min, 0° C.

Compounds AMFA4 and AMFA6 were synthesized following the synthetic routes described in FIG. 7. One of the main steps in the synthesis of these saccharide ligands was the introduction of the carboxylate function on the side chain of the mannose moieties. This functionalization is achieved by a Dess-Martin oxidation of alcohol 19 in position 6, followed by a Horner-Wadsworth-Emmons reaction using triethyl phosphonacetate anion. In this way, the unsaturated carboxylate 20 is obtained. Another critical step was the functionalization of the aglycone fragments with diethyl squarate or maleimide. This reaction was carried out on the fully deprotected compound 21 to provide AMFA4 and AMFA6 respectively (FIG. 7).

### Preparation of Compounds AMFA7 and AMFA8

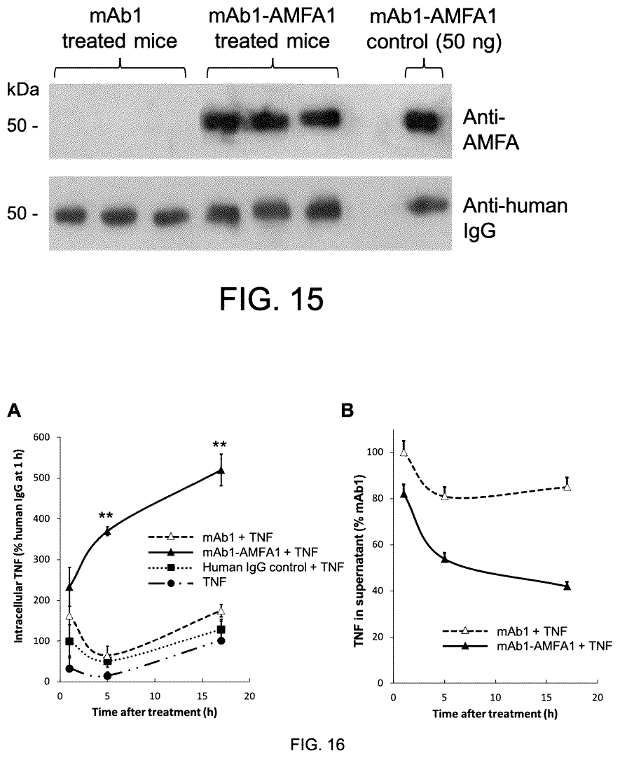

The process for preparing compound AMFA7 from compound 16, and compound AMFA8 from compound 21, respectively, is illustrated in FIG. 8. The conditions and reagents for step (i) are as follows: (i) 3-benzoylacrylic acid, N-methylmorpholine, isobutyl chloroformate, DMF, 1 h, −10° C. to RT. To prepare compounds AMFA7 and AMFA8, the same operating procedures used in the preparation of compounds AMFA3 and AMFA4 were followed, except for the last step. Indeed, the last step consists in the reaction between the amines of the respective intermediates 16 and 21 and 3-benzoylacrylic acid activated by isobutyl chloroformate in the presence of N-methylmorpholine (FIG. 8).

### Example 2: Synthesis of Conjugates of General Formula (I)

AMFA engineering has been validated on five different antibodies that have been coupled to AMFAs of formula (II):
two antibodies directed against Tumor Necrosis Factor alpha (TNF-alpha), called infliximab and adalimumab, and referred to in the application as mAb1 and mAb2 respectively;
an antibody directed against Vascular Endothelial Growth Factor (VEGF), called bevacizumab, referred to as mAb3;
an antibody against human epidermal growth factor receptor 2 (HER2), called trastuzumab, referred to as mAb4; and
an antibody against green fluorescent protein (GFP), referred to as Ab.

The grafting of AMFAs, for these non-limiting examples, can be carried out at:
the oligosaccharicic chain of the glycosidic part of the recombinant antibody (see points 1/to 5/below) with AMFA1, or
the amine function of the lysine residues of the peptide part of the recombinant antibody (see point 6/below) with AMFA4.
   1/ Grafting of AMFA1 onto the Oligosaccharidic Chains of the Glycosidic Portion of a Chimeric Anti-TNF-Alpha Antibody

AMFA1 coupling has been tested on a commercial mouse-human chimeric monoclonal antibody to human TNF-alpha, infliximab, defined as mAb1. A conjugate of formula (I) is formed which can indifferently be represented in the application as “mAb1-AMFA1” or “AMFA1-mAb1”. This conjugate comprises a part (mAb1) that binds to a specific target (such as an extracellular or membrane antigen) and a part (AMFA1) that binds to CI-M6PR. The mAb1 antibody was chosen as a model in the present application because it has oligosaccharidic chains on the constant regions that are very similar to those produced in humans. Coupling between AMFA1 and the mAb1 antibody is carried out according to the protocol described below.

In a first step, the mAb1 antibody is oxidized. Specifically, the mAb1 antibody and a solution of 1 mM sodium meta-periodate (NaIO₄) are reacted in 25.5 mM phosphate buffer pH 6.25 for 30 min at 4° C. in the dark. Glycerol (20 μL/mL) is added and incubated for 10 min at 4° C. to stop the reaction. Low-molecular-weight compounds are removed using a PD-10 desalting column pre-filled with Sephadex G-25 resin.

In a second step, AMFA1 is added to the oxidized antibody solution obtained previously, and the mixture is allowed to react under stirring for 2 h at 37° C. Finally, the samples are dialyzed overnight against phosphate buffer. The first step of the above protocol leads to the oxidation of the oligosaccharidic chains of the mAb1 antibody and generates aldehyde groups that covalently interact with the oxy-amine groups of AMFA1 to form an oxime bond. Furthermore, only the oligosaccharidic chains of the mAb1 antibody are remodeled. Indeed, the peptide part of mAb1 is not affected by the conditions used for AMFA1 grafting.

2/ Grafting of AMFA1 onto the Oligosaccharidic Chains of the Glycosidic Portion of a Humanized Anti-TNF-Alpha Antibody

The anti-TNF-alpha antibody, adalimumab, defined as mAb2, was tested and subjected to the same procedure as described in point 1/above in order to graft AMFA1 onto the oligosaccharide chains of the glycosidic part of the mAb2 antibody. A conjugate of formula (I) is formed, which can indifferently be represented in the application as “mAb2-AMFA1” or “AMFA1-mAb2”. This conjugate comprises a portion (mAb2) that binds to a specific target (such as an extracellular or membrane antigen) and a portion (AMFA1) that binds to CI-M6PR.

3/ Grafting of AMFA1 onto the Oligosaccharidic Chains of Glycosidic Part of a Humanized Anti-VEGF Antibody

The anti-VEGF antibody, bevacizumab, defined as mAb3, was tested and subjected to the same procedure as described in point 1/above, in order to graft AMFA1 onto the oligosaccharidic chains of the mAb3 antibody. A conjugate of formula (I) is formed, which can be indifferently represented in the application as “mAb3-AMFA1” or “AMFA1-mAb3”. This conjugate comprises a portion (mAb3) that binds to a specific target (such as an extracellular or membrane antigen) and a portion (AMFA1) that binds to CI-M6PR.

4/ Grafting of AMFA1 onto the Oligosaccharidic Chains of the Glycosidic Part of a Humanized Anti-HER2 Antibody

The anti-HER2 antibody, trastuzumab, defined as mAb4, was tested and subjected to the same procedure as that described in point 1/above, in order to graft AMFA1 onto the oligosaccharide chains of the mAb4 antibody. A conjugate of formula (I) was formed, which can indifferently be represented in the application by “mAb4-AMFA1” or “AMFA1-mAb4”. This conjugate comprises a portion (mAb4) that binds to a specific target (such as an extracellular or membrane antigen) and a portion (AMFA1) that binds to CI-M6PR.

5/ Grafting of AMFA1 onto the Oligosaccharide Chains of the Glycosidic Portion of an Anti-GFP Antibody

The anti-GFP antibody, defined as Ab, was tested and subjected to the same procedure as described in point 1/above in order to graft AMFA1 onto the oligosaccharide chains of the Ab antibody. A conjugate of formula (I) is formed, which can be represented indifferently as “Ab-AMFA1” or “AMFA1-Ab”. This conjugate comprises a part (Ab) that binds to a specific target (such as an extracellular or membrane antigen) and a part (AMFA1) that binds to CI-M6PR.

6/ Grafting of AMFA4 onto the Amine Function of Lysine Residues of the Peptide Part of a Humanized Anti-VEGF Antibody

Coupling between AMFA4 and the mAb3 antibody (bevacizumab) is carried out at the level of the mAb3 peptide chain, in particular at the lysine residues, according to the protocol described below. The mAb3 antibody solution and AMFA4 are reacted in phosphate buffer for 24 h at 37° C. with gentle agitation. Samples are dialyzed overnight against phosphate buffer, and a conjugate of formula (I) is formed, which can be represented as either “mAb3-AMFA4” or “AMFA4-mAb3”. This conjugate comprises a part (mAb3) that binds to a specific target (such as an extracellular or membrane antigen) and a part (AMFA4) that binds to CI-M6PR.

### Example 3: Study of Conjugates (I) and their Biological Properties

1/ Study of the Number of AMFA1 or AMFA4 Bound to a mAb1, mAb2, mAb3 or mAb4 Antibody Molecule

AMFA1-mAb1, AMFA1-mAb2, AMFA1-mAb3, AMFA1-mAb4 and AMFA4-mAb3 conjugates were analyzed by MALDI-TOF mass spectrometry using a RapifleX mass spectrometer (Bruker) in linear mode, in order to quantify the number of AMFA1 bound to an antibody molecule, according to the method adapted and described in Zhou, Q et al. 2013.

The molecular weights of each mAb1, mAb2, mAb3 and mAb4 antibody were measured before and after coupling with AMFA1 or AMFA4. Samples and standards were diluted 1:5 in 0.1% formic acid in water, then diluted 1:1 in saturated sinapic acid in 50% acetonitrile/0.1% TFA. One μL (microliter) of the mixture was applied to a target. Samples, corresponding references and BSA calibration control were analyzed in duplicate. The number of AMFA1 or AMFA4 grafted to mAb1, mAb2, mAb3 or mAb4 antibodies to obtain the conjugates of the invention was calculated based on the difference in molecular weights between the “AMFA-Antibody” conjugate and its corresponding control “Antibody”, then dividing the difference by the molecular weight of each AMFA.

Table 1 below shows MALDI-TOF analysis data for various batches of AMFA1-mAb1, AMFA1-mAb2, AMFA1-mAb3 and AMFA1-mAb4 conjugates as obtained in Example 2 (points 1/to 4/).

TABLE 1

| | | | | Average number of |
|---|---|---|---|---|
| | | | | AMFAs per |
| | | | | antibody ± SD |
| | | Sodium | | (standard deviation) |
| | Batch | metaperiodate | | (from 4 to 7 assays) |
| AMFA1-mAb1 | 1, 2 | 1 | mM | 3.3 ± 0.01 |
| AMFA1-mAb1 | 2, 3 | 0.2 | mM | 1 ± 0.1 |
| AMFA1-mAb2 | 1, 2, 3 | 1 | mM | 2.8 ± 0.3 |
| AMFA1-mAb3 | 1, 2 | 1 | mM | 5.8 ± 1 |
| AMFA1-mAb4 | 1, 2, 3 | 1 | mM | 3.0 ± 0.8 |

Table 2 below shows MALDI-TOF analysis data for various batches of AMFA4-mAb3 conjugates as obtained in Example 2 (point 6/).

TABLE 2

| | | | Average number of |
|---|---|---|---|
| | | | AMFAs per |
| | | Grafting | antibody ± SD |
| | Batch | method | (4 assays) |
| AMFA4-mAb3 | 1, 2 | Lysine-squarate | 1.9 ± 0.7 |

### Comments and Conclusion

The increase in molecular weight after grafting of AMFA1 or AMFA4 is an indication of the number of AMFAs coupled per antibody. The reproducibility of the grafting reaction is indicated by the low standard deviation of the number of AMFAs grafted between different assays. The data in Tables 1 and 2 demonstrate that AMFA1 and AMFA4 can be easily grafted onto mAb1, mAb2, mAb3 or mAb4, with high reproducibility standards, and that the number of AMFA1 or AMFA4 grafted onto each antibody is controlled by modulating the reaction conditions. Lower concentrations of the oxidizing agent, sodium metaperiodate, result in a lower grafting rate of AMFA1. Grafting of the AMFAs of the invention is reproducible and easily controlled according to the reaction conditions.

2/ Study of the Major Structural Modifications Associated with the Grafting of AMFA1 onto mAb1, mAb2, mAb3 or mAb4 Antibodies

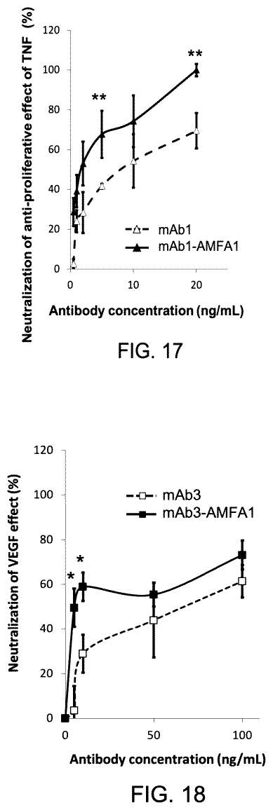

mAb1, mAb2, mAb3 and mAb4 antibodies alone or conjugated to AMFA1 (mAb1-AMFA1, mAb2-AMFA1, mAb3-AMFA1 and mAb4-AMFA1 (the mAb-AMFA conjugates)) were analyzed on non-denaturing and denaturing SDS-PAGE. Coomassie blue staining revealed a single band (under non-denaturing conditions) or two bands (under denaturing conditions) of the same molecular weight for all antibodies before and after coupling with AMFA1. Binding of AMFA1 to the antibody did not induce visible degradation or significant shifts in the molecular weight of the antibody heavy or light chains, due to the low number of grafted analogues. mAb-AMFA conjugates showed unchanged immunoreactivity to a second commercial anti-human IgG antibody in Western blot compared with mAbs. Hybridization with a rabbit anti-AMFA1 antibody (indicated as “anti-AMFA” in FIG. 9) confirmed the presence of AMFA1 on the heavy chain of AMFA1-linked antibodies (according to the protocols described in Basile I. et al., 2018). The results obtained are illustrated in FIG. 9.

Western blot analyses of the different antibodies demonstrate that oxidation does not alter the overall structure of the antibody, and that AMFA1 coupling is effective on the heavy chains of each mAb1, mAb2, mAb3 or mAb4 antibody, as demonstrated by the detection of AMFA1 by the rabbit anti-AMFA1 antibody. In conclusion, grafting AMFA1 to mAb1, mAb2, mAb3 or mAb4 antibodies does not modify the integrity of the antibody. Moreover, the detection of AMFA1 on the heavy chains of mAb1, mAb2, mAb3 and mAb4 antibodies is consistent with the grafting only on the oligosaccharidic chains carried by the heavy chains. The fact that the treatment with PNGase for the hydrolysis of the N-glycosylated chains enabled the detachment of AMFA1 from mAb1-AMFA1 indicates the grafting specificity 4 onto oligosaccharidic chains.

3/ Study of the Ability of mAb1-AMFA1 and mAb2-AMFA1 Conjugates to Bind to CI-M6PR

Affinity assays of the conjugates of the invention mAb1-AMFA1 and mAb2-AMFA1 for CI-M6PR were performed using biotinylated CI-M6PR (Basile et al, 2018). As used herein, the affinity of mAb1-AMFA1 and mAb2-AMFA1 conjugates for CI-M6PR means the ability of mAb1-AMFA1 and mAb2-AMFA1 conjugates to bind to CI-M6PR. Briefly, CI-M6PR, purified on a phosphomannane-sepharose affinity column, was biotinylated with biotin N-hydroxysuccinimide. Binding of biotinylated CI-M6PR (b-CI-M6PR) to pentamannose 6-phosphate (PMP) previously adsorbed on a microtiter plate was prevented by competition with increasing concentrations of mAb1-AMFA1 or mAb2-AMFA1 conjugates. The b-CI-M6PR bound to adsorbed PMP was then determined using streptavidin conjugated to the peroxidase and its substrate, o-phenylenediamine dihydrochloride (OPD), by optical density measurements. The 100% value corresponds to the concentration of b-CI-M6PR bound to PMP adsorbed in the absence of a competitor.

Table 3 below presents data from binding assays for natural M6P to b-CI-M6PR and for mAb1-AMFA1 and mAb2-AMFA1 conjugates to b-CI-M6PR. The studied conjugates comprise a portion (mAb1 or mAb2) that targets a target molecule (antigen) and a portion (AMFA1) that targets b-CI-M6PR. Affinity (expressed as the concentration required for 50% inhibition of the binding, IC₅₀) was determined by competitive assay with the pentamannose 6-phosphate ligand.

TABLE 3

| Ligand | Affinity for b-CI-M6PR (IC₅₀) |
|---|---|
| M6P | 2.10⁻⁵M |
| mAb1-AMFA1 | 6.10⁻⁵M |
| mAb2-AMFA1 | 1.7.10⁻⁵M |

### Comments and Conclusion

While the binding capacity of the mAb1 or mAb2 antibody alone to CI-M6PR is close to zero (less than 10% binding at a concentration range of 5.10⁸-2.10⁶ M), Table 3 shows that the mAb1-AMFA1 or mAb2-AMFA1 conjugate of the invention has a higher binding affinity for CI-M6PR than that of natural M6P for CI-M6PR. The conjugates of the invention thus acquire the ability to bind to CI-M6PR. In particular, the grafting of AMFA1 onto mAb1 and mAb2, respectively, enables the mAb1-AMFA1 and mAb2-AMFA1 conjugates of the invention to gain in affinity for CI-M6PR.

4/ Study of the Impact of AMFA1 Grafting on the Binding Affinity of mAb1 to mAb4 Antibodies to the Target Molecule, i.e. the Corresponding Antigen (Human TNF-Alpha, VEGF or HER2).

The affinity of mAb1, mAb2, mAb3, mAb4 antibodies alone, and mAb1-AMFA1, mAb2-AMFA1, mAb3-AMFA1 and mAb4-AMFA1 conjugates for target antigens (human TNF-alpha, VEGF or HER2) was assessed using an ELISA assay to determine antigen binding. By “affinity” of the antibody or conjugate to the antigen is meant the ability of said antibody or conjugate to bind to said antigen. Briefly, the antigen (commercial human TNF-alpha, VEGF or HER2) was adsorbed onto a microwell plate and incubated with the corresponding antibody (mAb1, mAb2, mAb3, mAb4) or conjugate (mAb1-AMFA1, mAb2-AMFA1, mAb3-AMFA1 and mAb4-AMFA1) respectively.

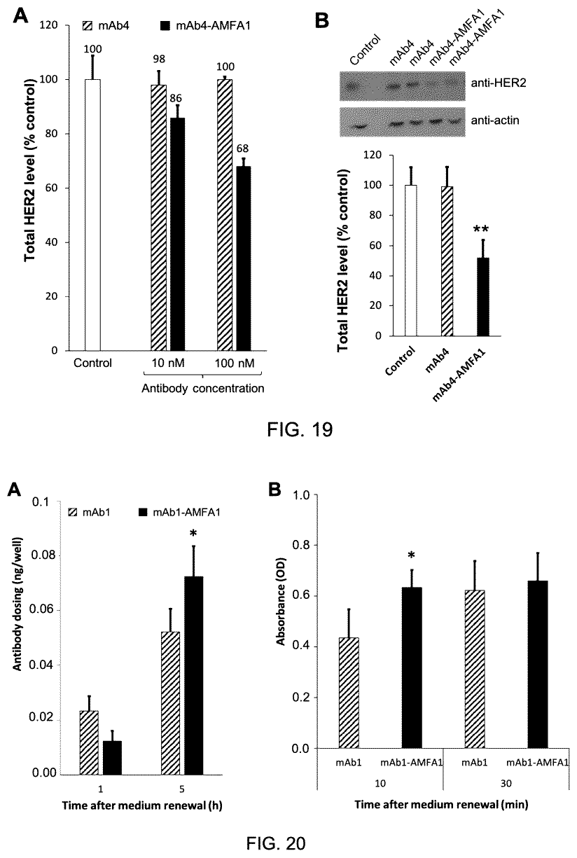

The level of antibodies or conjugates bound to the target of interest is determined by an anti-human IgG antibody coupled to horseradish peroxidase and the OPD substrate using optical density measurements. FIG. 10 shows that grafting AMFA1 onto mAb1, mAb2, mAb3 or mAb4 antibodies does not affect their affinity for their target antigen (human TNF-alpha, VEGF or HER2). The conjugates of the invention have an affinity for their antigen comparable to that of the antibodies alone.

5/ Stability Study of the mAb1-AMFA1 Conjugate of the Invention

mAb1-AMFA1 conjugates from different batches were incubated in acetate buffer (pH 5) or phosphate buffer (pH 7) for up to 12 days, then analyzed by MALDI-TOF to determine whether AMFA1 was still bound to the mAb1 antibody. The number of AMFA1 grafted to the mAb1 antibody was determined at day 0 and day 12 at pH 5 and 7. The results obtained are shown in Table 4 below.

TABLE 4

| | | | | Average number of |
|---|---|---|---|---|
| | | | | AMFAs per |
| | | | | antibody ± SD |
| | | Incubation | | (standard deviation) |
| | pH | time | | (2 to 4 assays) |
| AMFA1-mAb1 | 7 | 0 | day | 3.3 ± 0.1 |
| AMFA1-mAb1 | 5 | 12 | days | 2.0 ± 0.9 |
| AMFA1-mAb1 | 7 | 12 | days | 3.0 ± 0.1 |

### Comments and Conclusion

Table 4 shows that the number of AMFA1 grafted onto the mAb1 antibody is stable at two different pH levels, even after 12 days. AMFA1-mAb1 conjugates are therefore stable over time and at different pH levels.

6/ Study of Cellular Internalization of mAb1-AMFA1, mAb3-AMFA1 and Ab-AMFA1 Conjugates

Cellular internalization of the antibodies and conjugates of the invention was observed in rhabdomyosarcoma cells, T lymphocyte cells and breast cancer cells. Data were obtained by flow cytometry and total cell fluorescence assays. Rhabdomyosarcoma cells, T-lymphocyte cells and breast cancer cells were treated for 24 h or 48 h with a complex comprising mAb1-AMFA1 and a secondary fluorescent anti-human IgG antibody Alexa647 (FIGS. 11(A) and 12(A)). Prior to incubation with cells, the mAb1 antibody, the mAb1-AMFA1 conjugate and a non-specific control human IgG were incubated with the Alexa647 anti-human IgG fluorescent secondary antibody. Fluorescence in the cells was then analyzed by flow cytometry (FIG. 11(A)).

The data in FIG. 11(A) show significantly higher fluorescence (7.9, 11.3 and 11.8 times higher than control) in cells treated with mAb1-AMFA1. FIG. 11(B, C) shows the entry of mAb1 and mAb1-AMFA1, and mAb4 and mAb4-AMFA1 previously coupled to Alexa647 into T lymphocytes and SKOV3 ovarian cancer cells, respectively. FIG. 11 shows that AMFA1 enhances cellular uptake of the mAb1-AMFA conjugate compared with mAb1 alone. Adding AMFA1 to the mAb1 antibody significantly increased cellular internalization of the antibodies, and these results were confirmed by total cell fluorescence internalization experiments with mAb1, mAb1-AMFA1, mAb3 and mAb3-AMFA1 (FIG. 12). Rhabdomyosarcoma and breast cancer cells were treated for 24 h under the same conditions as described above. FIG. 12 shows that higher fluorescence is observed in cells treated with mAb1-AMFA1 versus mAb1 (breast cancer cells) and mAb3-AMFA1 versus mAb3 (rhabdomyosarcoma cells). Grafting of AMFA1 with mAb1 or mAb3 antibody significantly increased cellular internalization of antibodies.

These results were confirmed by confocal microscopy on live rhabdomyosarcoma and breast cancer cells (FIG. 13(A)). Cells were pre-incubated with 200 nM Ab antibody or Ab-AMFA1 conjugate (see Example 2, point 5/) and 400 nM green fluorescent protein, for 24 h. Higher fluorescence was observed with Ab-AMFA1 versus Ab alone in both cell types (fluorescent cell vesicles indicated by arrows in FIG. 13). Addition of an M6P excess (10 mM) to the cell medium to saturate the CI-M6PR binding sites showed that the internalization of Ab-AMFA1 into cells is significantly reduced in both cell types. In another experiment, mAb1 and mAb1-AMFA1 were coupled to the Alexa467 fluorophore to measure their internalization in Jurkat cells (T lymphocytes). The increased internalization of mAb1-AMFA1 compared to mAb1, and the reversion of mAb-AMFA1 entry by the addition of 30 mM AMFA1 excess are described and quantified in FIG. 13(B, C). More broadly, these results demonstrate that grafting AMFAs onto antibodies induces an increase in cellular internalization via the CI-M6PR-mediated transport pathway.

7/ Study of the Pharmacokinetic Profile of the mAb1-AMFA1 and mAb2-AMFA1 Conjugates of the Invention

The effect of AMFA1 grafting on mAb1 and mAb2 antibodies targeting TNF-alpha was studied by comparing the pharmacokinetic profile of mAb1 or mAb2 with that of the corresponding mAb1-AMFA1 or mAb2-AMFA1 conjugates. mAb1 or mAb2 antibodies and the corresponding mAb1-AMFA1 or mAb2-AMFA1 conjugates were administered to 10-week-old male C57BL/6J mice in a single intravenous bolus injection of 4 mg/kg diluted in physiological saline solution. Mice were weighed before injection and throughout the experiment to monitor their health. Blood samples taken at 4 and 1, 2, 3, 7, 10, 14, 17 and 21 days post-injection were collected in tubes containing heparin and centrifuged at 3,000 g for 15 min, and the plasma was stored at −20° C. until measurement. Mice were sacrificed 21 days after injection. No adverse clinical events were observed in any of the four treatment groups.

Plasma concentrations of mAb1, mAb2, mAb1-AMFA1 and mAb2-AMFA1 were measured using ELISA techniques. Briefly, recombinant human TNF-alpha was adsorbed onto a Maxisorp microwell plate and recognized by mAb1, mAb2, mAb1-AMFA1 or mAb2-AMFA1 present in plasma samples. The therapeutic antibody was detected using an HRP-conjugated anti-human IgG antibody specific for the Fc fragment, which was then quantified by a colorimetric assay with the OPD substrate and absorbance measure at 450 nm. The results obtained are shown in FIG. 14. The pharmacokinetic profiles of mAb1 compared with mAb1-AMFA1 (A) and of mAb2 compared with mAb2-AMFA1 (B) detected in plasma samples from treated animals, expressed as a percentage of the values at 4 h, are similar. No significant difference in antibody half-life was observed with the grafting of AMFA1 onto mAb1 or mAb2. In conclusion, grafting AMFA1 onto mAb1 and mAb2 antibodies targeting the TNF-alpha antigen does not alter the pharmacokinetic profile of the antibodies.

8/ In Vivo Stability Study of the mAb1-AMFA1 Conjugate of the Invention

The in vivo stability of AMFA1 binding to the therapeutic mAb1 antibody was assessed by analyzing the presence of AMFA1 in plasma samples collected at 21 days from mice treated with mAb1-AMFA1 or mAb1 as described in 7/above. mAb1 or mAb1-AMFA1 were first isolated from plasma samples by binding for 24 h to an anti-human IgG antibody specific for the Fc fragment previously adsorbed onto a microwell plate. As a loading control, 50 ng of mAb1-AMFA1 diluted in phosphate buffer were also incubated. After washing, the bound antibodies were analyzed by Western blot for the presence of AMFA1 using a polyclonal anti-AMFA1 antibody (represented as “anti-AMFA” in FIG. 15) and with an anti-human IgG antibody to control the amount of mAb1 or mAb1-AMFA1 present at 21 days. FIG. 15 provides evidence that AMFA1 is still conjugated to the mAb1 therapeutic antibody at 21 days. FIG. 15 shows that both mAb1 and mAb1-AMFA1 are detected in the plasma of treated mice after 21 days at a similar level to that of the 50 ng mAb1-AMFA1 control, as confirmed by hybridization with anti-human IgG. The majority of AMFA1 is still bound to mAb1, the signal intensity of the band being comparable to that of mAb1-AMFA1 used as a control.

9/ Study of the Ability of the mAb1-AMFA1 Conjugate to Increase Cellular Internalization of Human TNF-Alpha

This study shows that human TNF-alpha antigen is degraded over time after internalization in cells with the mAb1-AMFA1 conjugate. The degradation of human TNF-alpha was studied in human T-lymphocyte cells. Cells were treated with 5 ng/mL human TNF-alpha and 20 ng/mL mAb1 or mAb1-AMFA1, for 1 h, 5 h and 17 h. Cell lysates were analyzed by Western blot for intracellular human TNF-alpha. For the mAb1-AMFA1 conjugate, the data show an increase in the level of human TNF-alpha up to 17 h (FIG. 16(A)). In contrast, only a slight increase in intracellular human TNF-alpha is observed with the mAb1 antibody. The data demonstrate that human TNF-alpha is better internalized with mAb1-AMFA1 than with mAb1 antibody. The amounts of TNF-alpha measured by Western blot in the culture medium during treatment with mAb1-AMFA1 and mAb show that mAb1-AMFA1 causes a greater decrease in TNF-alpha (FIG. 16(B)). This strong decrease in the external medium is correlated with an increased cellular uptake of TNF-alpha.

10/ Study of the Ability of the mAb1-AMFA1 Conjugate to Reduce the Biological Effect of Human TNF-Alpha

This study shows that the mAb1-AMFA1 conjugate not only improves internalization in the cell, but also reduces the biological effect to a greater extent than the antibody alone. L929 mouse fibroblast cells were treated with 5 ng/mL human TNF-alpha and increasing doses of mAb1 or mAb1-AMFA1. Since human TNF-alpha induces cytotoxicity in L929 cells, the rate of living cells after 48 h treatment with mAb1 or mAb1-AMFA1 was analyzed by the MTT method. FIG. 17 shows the neutralizing effect of mAb1 or mAb1-AMFA1 on human TNF-alpha toxicity. mAb1-AMFA1 neutralizes human TNF-alpha toxicity at much lower doses (4 to 5 times) than mAb1. In conclusion, mAb1-AMFA1 conjugates induce a more effective dose-dependent neutralization of human TNF-alpha-induced cell growth inhibition than mAb1 alone.

11/ Study of the Ability of the mAb3-AMFA1 of the Invention to Reduce the Effect of Human VEGF on the Growth of HUVEC Cells

In this study, it was shown that the mAb3-AMFA1 conjugate, in addition to an improved internalization into the cell, allows a greater reduction in the biological effect than that observed with the mAb3 antibody alone. HUVEC human umbilical vein endothelial cells were treated with 50 ng/mL human VEGF and increasing doses of mAb3 or mAb3-AMFA1. Since human VEGF induces proliferation of HUVEC cells, the live cell count at 72 h on cells treated with mAb3 or mAb3-AMFA1 was analyzed by the MTT method. FIG. 18 shows that inhibition of the mitogenic effect of VEGF in HUVEC cells is greater with the mAb3-AMFA1 conjugate than with the antibody alone. At low doses, the mAb3-AMFA1 conjugate is twice as effective as the mAb3 antibody alone. The mAb3-AMFA1 conjugate is effective at much lower doses (10 times) than mAb3.

### 12/ Study of the Ability of AMFA1-mAb4 Conjugates to Reduce the Amount of HER2

SK-BR-3 breast cancer cells were treated with 10 or 100 nM mAb4 or mAb4-AMFA1 for 24 h. The cells were then fixated and the amount of target protein measured using a colorimetric method based on indirect ELISA. The HER2 protein is recognized by a specific antibody, whose Fc moiety will in turn be recognized by a secondary antibody coupled to peroxidase (HRP). FIG. 19(A) shows the decrease in HER2 protein after treatment with mAb4-AMFA1 compared with mAb4 alone in SK-BR3 cells. Increasing doses of mAb4-AMFA1 result in a greater decrease in cellular HER2 concentration compared with the same doses of mAb4. BT474 breast cancer cells were treated with 5 nM mAb4 or mAb4-AMFA1 for 5 h. Total HER2 was quantified by Western blot. After 5 h treatment with mAb4-AMFA1, the amount of HER2 is strongly decreased, whereas treatment with mAb4 is not effective (FIG. 19(B)).

13/ Extracellular Recycling of AMFA1-mAb1 Conjugates after Internalization in T Lymphocytes

Extracellular recycling of internalized mAb1 antibody and mAb1-AMFA1 conjugate was demonstrated by experiments involving 5 h cellular entry of antibodies into immortalized T-lymphocyte cells (Jurkat), followed by a change of medium to an antibody-free medium and a 1 and 5 h period of release of previously internalized antibodies into the culture medium (FIG. 20(A)). In another experiment, cells are treated for 30 min with antibodies and, after changing the medium, the release of internalized antibodies is measured after 10 and 30 min (FIG. 20 (B)). Concentrations of mAb1 and mAb1-AMFA1 were measured using ELISA as described in 7/above. FIG. 20 shows that extracellular recycling of mAb1-AMFA1 conjugates is comparable to that of mAb1 alone. These results indicate that mAb1-AMFA1 is normally recycled out of the cell after its strong cellular internalization mediated by CI-M6PR.

The present disclosure is not limited to the examples described above, by way of example only, but embraces all variants which may be envisaged by a person skilled in the art in the context of the protection sought.

### LIST OF CITED DOCUMENTS

### Patent Documents

For all intents and purposes, the following patent documents are cited:
patcit1: EP 2 448 600 B1;
patcit2: EP 3 350 192 B1;
patcit3: PCT/US2019/067228;
patcit4: PCT/IB2021/050922.

### Non-Patent Literature

For all intents and purposes, the following non-patent elements are cited:
nplcit1: Gary-Bobo et al. Curr. Med. Chem. 2007, 14, 2945-2953;
nplcit2: El Cheikh K. et al., Angew. Chem. Int. Ed. 2016, 55, 52016;
nplcit3: Zhu Y., et al. Mol Ther. 2009, 17, 954-963;
nplcit4: Basile I. et al., J. Control. Release, 2018, 10, 269, 15-23;
nplcit5: Zhou, Q et al., Bioconjugate Chem. 2013, 24, 12, 2025-2035.

1-14. (canceled)

15. A conjugate having a general formula (I) 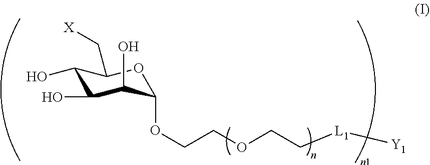
wherein:
X is a phosphonate group —CH₂—P(O)(OZ)₂ or carboxylate group —CH₂—C(O)(OZ) with Z representing independently from each other H, Na, Li, K or NH₄;
n is an integer ranging from 0 to 2;
L₁ is a radical selected from the group comprising *—O—N═**, 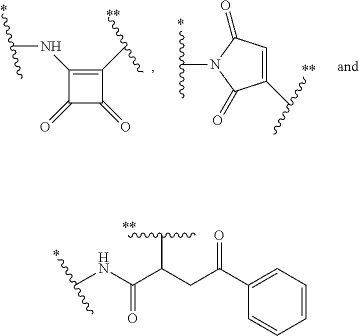 [SMILES: *Nc1c(*)c(=O)c1=O ; *C1=CC(=O)N(*)C1=O ; *NC(=O)C(*)CC(=O)c1ccccc1]
with * indicating a point of attachment of L₁ to the (O—CH₂—CH₂)ₙ group and ** indicating a point of attachment of L₁ to Y₁;
n₁ is an integer ranging from 1 to 20, and preferably from 1 to 10,
Y₁ represents an antibody Y or an antibody fragment Y comprising at least one antigen-binding domain,
said Y₁ forming n₁ covalent bond(s) with L₁.

16. The conjugate according to claim 15, wherein Y₁ is represented by a group L₂-Y′₁ in which Y′₁ represents the antibody Y or antibody fragment Y linked to L₁ via radical L₂ with L₂ chosen from the group comprising ═CH—, —NH— and —S—, it thus being possible for said Y₁ to be represented by ═CH—Y′₁, —NH—Y′₁ and —S—Y′₁.

17. The conjugate according to claim 15, wherein n₁ is an integer equal to 1 and in that said conjugate of formula (I) is selected from the group comprising: 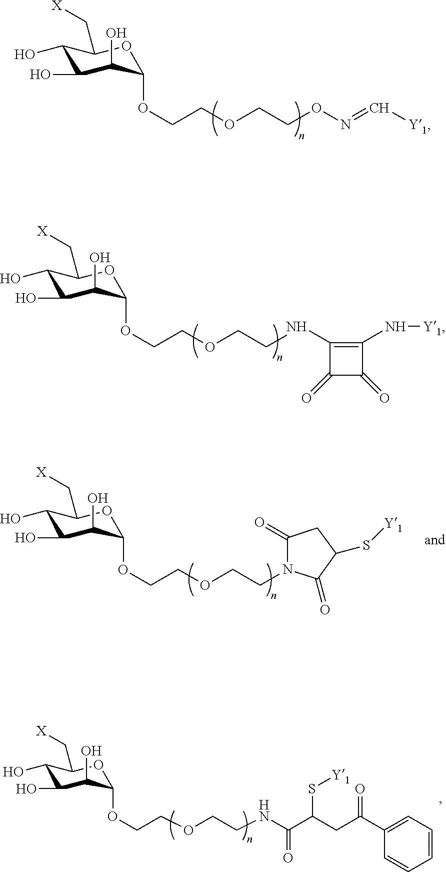
with X, n and Y′₁ as defined in claim 1 or 2.

18. The conjugate according to claim 15, wherein Y₁ represents a therapeutic or diagnostic antibody Y.

19. The conjugate according to claim 18, wherein Y₁ represents an antibody Y selected from the group comprising monoclonal antibodies, chimeric antibodies, human or humanized antibodies, heavy- and light-chain antibodies, single-chain antibodies, bispecific or multispecific antibodies and nanobodies.

20. The conjugate according to claim 19, wherein Y₁ represents an antibody Y which is an immunoglobulin of the IgG type, in particular of the lgG1, lgG2, lgG3 or lgG4 subtype, or an immunoglobulin of the IgE, IgD, IgA or IgM type.

21. The conjugate according to claim 15, wherein the conjugate has an IC₅₀ affinity towards the cation-independent mannose 6-phosphate receptor (CI-M6PR) ranging from 10⁻⁴ M to 10⁻⁹ M, and preferably ranging from 10⁻⁵ M to 10⁻⁸ M.

22. A process for preparing the conjugate of formula (I) as defined in claim 15, wherein:
an antibody Y or an antibody fragment Y comprising at least one antigen-binding domain is reacted with
n₁ compound(s) corresponding to general formula (II) 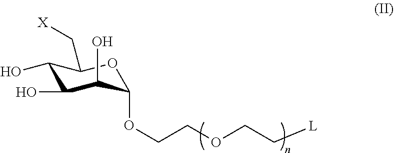
wherein X, n and n₁ are as defined in claim 1,
L is a reactive chemical group selected from the group comprising —O—NH₂, 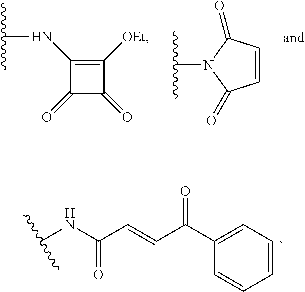 [SMILES: *Nc1c(OCC)c(=O)c1=O ; *N1C(=O)C=CC1=O ; *NC(=O)/C=C/C(=O)c1ccccc1]
said group L of compound (II) forming n₁ covalent bond(s) with n₁ functional group(s) carried by said antibody Y or antibody fragment Y.

23. The process according to claim 22, wherein the compound (II) is selected from the group comprising

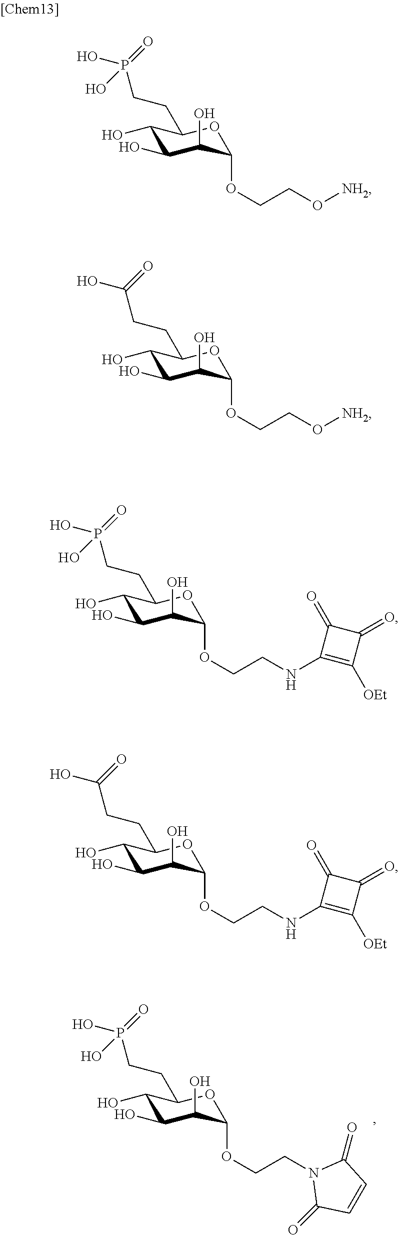

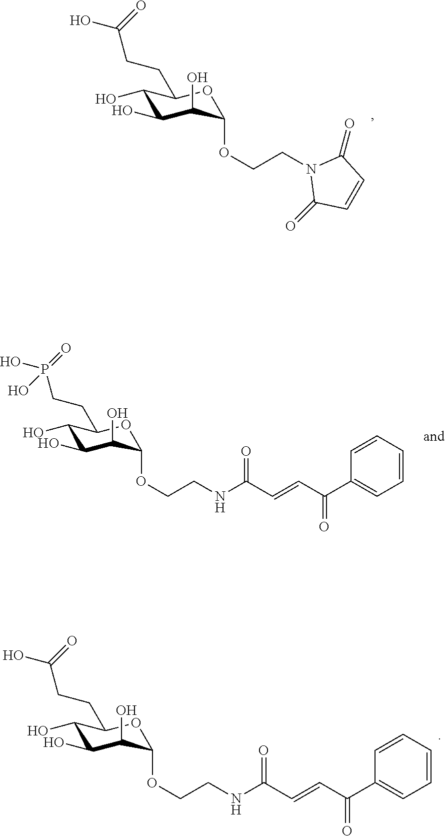

24. A pharmaceutical composition comprising:
the conjugate as defined in claim 15, and
at least one pharmaceutically acceptable excipient.

25. A medicine comprising the conjugate as defined claim 15 or a pharmaceutical composition comprising the conjugate and at least one pharmaceutically acceptable excipient.

26. A method of therapeutic treatment for a subject in need thereof, comprising administering to the subject a therapeutically effective amount of the conjugate as defined as defined claim 15, wherein Y₁ is a therapeutic antibody or a therapeutic antibody fragment, or a pharmaceutical composition comprising the conjugate and at least one pharmaceutically acceptable excipient.

27. A diagnostic method of detecting a target molecule in a subject comprising administering to the subject a conjugate as defined as defined claim 15, wherein Y₁ is a diagnostic antibody or a diagnostic antibody fragment targeting the molecule of interest, or a pharmaceutical composition comprising the conjugate and at least one pharmaceutically acceptable excipient.

28. The medicine of claim 25, wherein the conjugate or the pharmaceutical composition is in a form suitable for parenteral, intravenous or subcutaneous administration.

29. The method of therapeutic treatment of claim 26, wherein the conjugate or pharmaceutical composition is administered by parenteral, intravenous or subcutaneous administration.

30. The diagnostic method of claim 27, wherein the conjugate or pharmaceutical composition is administered by parenteral, intravenous or subcutaneous administration.
